(19) United States
(12) Patent Application Publication
(10) Pub. No.: US 2013/0053853 A1
(43) Pub. Date: Feb. 28, 2013

(54) DEVICES, METHODS AND SYSTEMS FOR NEURAL LOCALIZATION

(71) Applicants: Gregory P. SCHMITZ, Los Gatos, CA (US); Michael P. WALLACE, Pleasanton, CA (US); Jeffery L. BLEICH, Palo Alto, CA (US); Eric C. MILLER, Los Gatos, CA (US)

(72) Inventors: Gregory P. SCHMITZ, Los Gatos, CA (US); Michael P. WALLACE, Pleasanton, CA (US); Jeffery L. BLEICH, Palo Alto, CA (US); Eric C. MILLER, Los Gatos, CA (US)

(21) Appl. No.: 13/662,247

(22) Filed: Oct. 26, 2012

Related U.S. Application Data
(62) Division of application No. 12/352,385, filed on Jan. 12, 2009.
Application No. 12/352,385 is a continuation-in-part of application No. 12/060,229, filed on Mar. 31, 2008, now Pat. No. 7,959,577.
Application No. 12/060,229 is a continuation of application No. 13/619,930, filed on Sep. 14, 2012.
Application No. 13/619,930 is a continuation of application No. 13/090,944, filed on Apr. 20, 2011, now Pat. No. 8,303,516.
Application No. 13/090,944 is a division of application No. 12/060,229, filed on Mar. 31, 2008, now Pat. No. 7,959,577.
(60) Provisional application No. 61/020,670, filed on Jan. 11, 2008.
(60) Provisional application No. 61/017,512, filed on Dec. 28, 2007.
(60) Provisional application No. 61/020,670, filed on Jan. 11, 2008.
(60) Provisional application No. 61/017,512, filed on Dec. 28, 2007.
(60) Provisional application No. 60/976,029, filed on Sep. 28, 2007.
(60) Provisional application No. 60/970,458, filed on Sep. 6, 2007.

(51) Int. Cl. A61B 17/16; A61B 17/28; A61B 17/00

(52) U.S. Cl. 606/83; 606/1; 606/205

(57) ABSTRACT

Described herein are tissue manipulation devices having a tight bipole network. In particular, described herein are smart tools such as rongeurs configured to sense the presence of a nerve or portion of nerve. Tissue may be cut (or otherwise manipulated) by using a tool having a tight bipolar network to sense when a nerve or portion of a nerve is in the tool prior to cutting.

Also described are systems for determining if a nerve is nearby an insertable tool. These systems typically include a tool with a neurostimulation electrode, an accelerometer configured to detect muscle twitch, and a feedback controller to provide feedback indicating if the tool is near a nerve. Methods of controlling insertion of a tool using feedback from such a system are also described.

### CROSS-REFERENCE TO RELATED APPLICATIONS

This application is a divisional of U.S. patent application Ser. No. 12/352,385, titled “DEVICES, METHODS AND SYSTEMS FOR NEURAL LOCALIZATION,” filed on Jan. 12, 2009, now U.S. Patent Publication No. US-2009-0171381-A1, which claims priority to U.S. Provisional Patent Application Ser. No. 61/020,670, titled “DEVICES AND METHODS FOR TISSUE LOCALIZATION AND IDENTIFICATION,” filed on Jan. 11, 2008. Each of these patent applications is herein incorporated by reference in its entirety.

U.S. patent application Ser. No. 12/352,385 also claims priority as a continuation-in-part of U.S. patent application Ser. No. 12/060,229, titled “METHOD, SYSTEM, AND APPARATUS FOR NEURAL LOCALIZATION,” filed on Mar. 31, 2008, now U.S. Pat. No. 7,959,577 B2, which claims priority to U.S. Provisional Patent Application Ser. No. 61/017,512 titled “METHOD, SYSTEM, AND APPARATUS FOR TISSUE LOCALIZATION AND IDENTIFICATION,” filed on Dec. 28, 2007. Each of these patent applications is herein incorporated by reference in its entirety.

This application is a continuation of U.S. patent application Ser. No. 13/619,930, titled “METHOD, SYSTEM AND APPARATUS FOR NEURAL LOCALIZATION,” filed on Sep. 14, 2012, which is a continuation of U.S. patent application Ser. No. 13/090,944, titled “METHOD, SYSTEM AND APPARATUS FOR NEURAL LOCALIZATION,” filed on Apr. 20, 2011, now Publication No. US-2011-0196257-A1, which is a divisional of U.S. patent application Ser. No. 12/060,229, titled “METHOD, SYSTEM AND APPARATUS FOR NEURAL LOCALIZATION,” filed on Mar. 31, 2008, now U.S. Pat. No. 7,959,577, which claims priority to U.S. Provisional Patent Application Nos. 61/020,670, titled “DEVICES AND METHODS FOR TISSUE LOCALIZATION AND IDENTIFICATION,” filed on Jan. 11, 2008; 61/017,512, titled “METHOD, SYSTEM AND APPARATUS FOR TISSUE LOCALIZATION AND IDENTIFICATION,” filed on Dec. 28, 2007; 60/976,029, titled “METHOD AND APPARATUS FOR NEURAL LOCALIZATION,” filed on Sep. 28, 2007; and 60/970,458, titled “NERVE TISSUE LOCALIZATION SYSTEM,” filed Sep. 6, 2007. Each of these applications is herein incorporated by reference in its entirety.

### INCORPORATION BY REFERENCE

All publications and patent applications mentioned in this specification are herein incorporated by reference in their entirety, as if each individual publication or patent application was specifically and individually indicated to be incorporated by reference in its entirety.

### BACKGROUND

Many types of surgical intervention require manipulation of one or more medical devices in close proximity to a nerve or nerves, and therefore risk damage to the nerve tissue. For example, medical devices may be used to cut, extract, suture, coagulate, or otherwise manipulate tissue including or near neural tissue. It would therefore be beneficial to precisely determine the location and/or orientation of neural tissue when performing a medical procedure.

Knowing the location or orientation of a nerve in relation to a medical device (e.g., a probe, retractor, scalpel, etc.) would enable more accurate medical procedures, and may prevent unnecessary damage to nearby nerves. Although systems for monitoring neural tissue have been described, these systems are relatively imprecise. Further, many of these systems require large current densities (which may also damage tissue) and may be severely limited in their ability to accurately guide surgical procedures. For example, in many such systems a current is applied from an electrode (e.g., a needle electrode) in order to evoke an efferent muscular response such as a twitch or EMG response. Such systems typically broadcast, via the applied current, from the electrode and the current passes through nearby tissue until it is sufficiently near a nerve that the current density is adequate to depolarize the nerve.

Because the conductance of biological tissue may vary between individuals, over time in the same individual, and within different tissue regions of the same individual, it has been particularly difficult to predictably regulate the applied current. Furthermore, the broadcast fields generated by such systems are typically limited in their ability to spatially resolve nerve location and/or orientation with respect to the medical device.

For example, U.S. Patent Publication No. US-2005-0075578 to Gharib et al. and U.S. Patent Publication No. US-2005-0182454 to Gharib et al. describes a system and related methods to determine nerve proximity and nerve direction. Similarly, No. U.S. Pat. No. 6,564,078 to Marino et al. describes a nerve surveillance cannula system and U.S. Patent Publication No. US-2007-016097 to Farquhar et al. describes a system and method for determining nerve proximity and direction. These devices generally apply electrical current to send current into the tissue and thereby depolarize nearby nerves. Although multiple electrodes may be used to stimulate the tissue, the devices, systems and methods described are do not substantially control the broadcast field. Thus, these systems may be limited by the amount of current applied, and the region over which they can detect nerves.

Thus, it may be desirable to provide devices, systems and methods that controllably produce precise electrical broadcast fields in order to stimulate adjacent neural tissue, while indirectly or directly monitoring for neural stimulation (e.g. EMG, muscle movement, or SSEP), and thereby accurately determine if a nerve is in close proximity to a specified region of the device.

### SUMMARY OF THE DISCLOSURE

Described herein are medical devices for insertion into tissue that include a tight bipole network configured to detect nerve tissue immediately adjacent to the tissue manipulation region of the device. These medical devices may be referred to as “smart tools” because they can sense, and in some variations react to, the presence of nerve tissue. For example, described herein are rongeur devices including a tight bipole network. The tight bipole network is part of the tissue receiving portion of the rongeur, and is arranged so that it emits a broadcast field (e.g., current) that will stimulate a nerve that is present in the tissue receiving portion of the rongeur. The device is configured so that the broadcast field will not extend substantially beyond the tissue receiving portion, therefore providing specificity. The tight bipole network may also be arranged so it extends along the length of the tissue manipulation region of the medical device.

For example, described herein are tissue manipulation devices that can detect the presence of a nerve in a tissue to be manipulated by the device. These devices may include: a tissue receiving portion including a first tissue receiving surface and a second tissue receiving surface, wherein the first tissue receiving surface is configured to move relative to the second tissue receiving surface to engage tissue within the tissue receiving portion; and a tight bipole network in communication with the tissue receiving portion, wherein the tight bipole network is configured to emit a broadcast field that is limited to the tissue receiving portion and sufficient to stimulate a nerve within the tissue receiving portion.

The tissue manipulation device may be any device that includes a tissue receiving portion which can include a tight bipole network. For example, a tissue manipulation device may include a rongeur, a scissor, a clam, a tweezers, or the like. Rongeurs are of particular interest and are described in greater detail below, although much of this description may be applied to other tissue manipulation devices as well. A tissue manipulation device may be a tissue modification device. In general, a tissue manipulation device may include an elongate device (including a probe) that can be inserted into a patient, either in an open procedure or a percutaneous procedure. Thus, it may include a handle and/or an elongate body.

The tissue receiving portion of the tissue manipulation device may be a cavity or opening on the device into which tissue may fit or be placed. The tissue receiving portion may be static (e.g., a fixed size and/or shape), or it may be dynamic. For example, the tissue receiving portion may be made smaller to clamp or cut tissue. The tissue receiving portion may be located on the distal end, or near the distal end, of a device. In some variations, the tissue receiving portion opens from a side of the device that is proximal to the distal end of the device. The tissue receiving portion may be configured as a jaw.

As mentioned above, the tissue manipulation device may include a handle proximal to the tissue receiving portion. The handle may include a control for moving the first tissue receiving surface and/or the second tissue receiving surface. Any appropriate control may be used, e.g., knob, lever, dial, slider, etc. The tissue manipulation device may also include an elongate body extending proximally to the tissue receiving portion. This elongate body may be rigid, flexible, steerable, or capable of being made rigid or flexible along all or a portion of its length (e.g., by tensioning/un-tensioning an internal member, or by adding or removing a stiffening member, by inflating or deflating a stiffening bladder or the like).

The second tissue receiving surface may be movable or not movable. For example, the second tissue receiving surface may be formed from the elongate body of the device.

Tight bipole networks are described in greater detail below. In general, a tight bipole network includes at least one bipole pair of electrodes that are sufficiently close so that the current flowing between them forms a broadcast field that is very limited, allowing the tight bipole network to stimulate (and therefore allow detection of) nerves that are in the immediate region of the bipole network (e.g., adjacent to or contacting). A tight bipole network may include a plurality of anodes and cathodes that are arranged within the tissue receiving portion. Tight bipole network may include a plurality of anodes and cathode pairs that are arranged to form an effectively continuous bipole field within the tissue receiving portion. For example, a line of anodes and cathodes (which may be alternating) may be arranged down the length of the tissue receiving portion. In some variations, a line of cathodes and a line of anodes may be formed by creating openings (vias) to a wire or length of cathode extending proximally and a wire or length of anode extending proximally.

As mentioned, the tissue manipulation device may be configured as a rongeur and the first tissue receiving surface may be configured to move relative to the second tissue receiving surface to cut tissue within the tissue receiving portion. Other examples of rongeurs are described herein.

For example, also described herein are rongeur devices for cutting tissue that can detect the presence of a nerve in the tissue to be cut. A rongeur device may comprise: a jaw having a tissue receiving portion, the tissue receiving portion including a first tissue receiving surface and a second tissue receiving surface, wherein the first tissue receiving surface is configured to move towards the second tissue receiving surface to cut tissue within the tissue receiving portion; and a tight bipole network on the jaw configured to emit a broadcast field that is limited to the tissue receiving portion and sufficient to stimulate a nerve within the tissue receiving portion.

As with any of the tissue manipulation devices described, a rongeur device may include a handle, and/or an elongate body, wherein the jaw is located at the distal region of the elongate body. In some variations, the second tissue receiving surface is not movable. As described above, the tight bipole network comprises a bipole pair, and in some variations, the tight bipole network comprises a plurality of anodes and cathodes arranged within the tissue receiving portion. The tight bipole network may comprise a plurality of anodes and cathodes configured to form an effectively continuous bipole field within the tissue receiving portion.

Also described herein are rongeur devices for cutting tissue that can detect the presence of a nerve in the tissue to be cut, the rongeur device comprising: a handle; an elongate body extending distally from the handle along a longitudinal axis; a tissue receiving portion near the distal end of the elongate body, the tissue receiving portion including a first tissue receiving surface and a second tissue receiving surface, wherein the first tissue receiving surface is configured to move longitudinally towards the second tissue receiving surface to cut tissue within the tissue receiving portion; and a tight bipole network in communication with the tissue receiving portion wherein the tight bipole network is configured to emit a broadcast field that is limited to the tissue receiving portion and sufficient to stimulate a nerve within the tissue receiving portion.

Methods of using these tissue manipulation devices are also described. In general, the method of using a tissue manipulation device includes placing a tissue within the tissue receiving portion of the tissue manipulation device, energizing a tight bipole network to emit a broadcast field that is limited to the tissue receiving portion, and determining if a nerve or portion of a nerve is within the tissue receiving portion.

For example, described herein are methods of cutting tissue using a rongeur device capable of determining if a nerve is present in the tissue to be cut. These methods typically include the steps of placing tissue within a tissue receiving portion of the rongeur device, energizing a tight bipole network to emit a broadcast field that is substantially limited to the tissue receiving portion, determining if a nerve or a portion of a nerve is present in the tissue receiving portion of the rongeur device, and cutting the tissue within the tissue receiving portion of the rongeur device.

The step of energizing the tight bipole network may include applying energy to a plurality of bipole pairs in communication with the tissue receiving portion of the rongeur device. For example, energizing the tight bipole network comprises emitting an effectively continuous bipole field within the tissue receiving portion of the rongeur device.

The step of determining if a nerve or portion of a nerve is present may be performed in any appropriate way. Generally, this may include observing either the electrical activity of the nerve directly (e.g., by monitoring downstream electrical activity) or by monitoring the activity of the target of the nerve. In some variations, this means observing muscle activity, when the nerve(s) stimulated by the tight bipole network enervate a muscle or muscles. For example, activation of a nerve may be observed by detecting EMG (electromyogram) activity, or by observing/monitoring muscle twitch. This observation may be correlated with the timing of stimulation of the tight bipolar pair.

The step of cutting may include actuating the handle of the rongeur device to move a first tissue receiving surface of the tissue receiving portion of the rongeur device towards a second tissue receiving surface. In general, the tissue may be cut if a nerve or portion of a nerve is not present in the tissue receiving portion of the rongeur device.

In general, an accelerometer-based device or system may be used to determine stimulation of a nerve to determine proximity of the nerve to a neurostimulation electrode (including a tight bipole network) on a tool that is inserted into a patient. For example, an accelerometer may be placed on the patient to detect muscle twitch due to stimulation from a neurostimulation electrode. The signal from the accelerometer may be filtered (e.g., to remove low-frequency movement artifact), and may be coordinated with the stimulation by the neurostimulation electrode (e.g., time-synchronized). The use of an accelerometer as described herein may be advantageous over most currently used EMG type systems. For example, an accelerometer-based system may eliminate the need for a trained EMG technician.

The accelerometer may be disposable or re-usable. For example, in a disposable configuration the accelerometer may be secured to the patient and connected to a feedback controller that receives signals from the accelerometer and/or the stimulator controlling the neurostimulation electrode. The feedback controller may analyze the signal and provide an output from the accelerometer. Any appropriate output may be used (e.g., visual, audible, etc.). For example, a display may be used to indicate stimulation of a nerve by the neurostimulation electrode.

In some variations, the output may be feed back into the control of the tool that is inserted into the body. For example, when the tool is a cutting device (e.g., a rongeur, etc.), feedback from the feedback controller indicating the presence of a nerve may prevent the device from cutting. In some variations, when the tool is a probe, catheter, or the like, the feedback may be used to steer the tool. Any appropriate tool may be used, including tissue manipulation devices as described above, but also including other insertable tools (and not limited to just tissue manipulation tools like rongeurs). For example a tool may be an implant, such as a screw.

Thus, described herein are systems for determining if a nerve is nearby an insertable tool. Such systems may include: an insertable tool having a first surface comprising a neurostimulation electrode configured to detect proximity to a nerve; an accelerometer to detect muscle movement upon stimulation of a nerve by the neurostimulation electrode; and a feedback controller configured to receive input from the accelerometer and determine activation of a nerve by the neurostimulation electrode, wherein the feedback controller is further configured to provide feedback to tool to control operation of the tool. As mentioned above, example of tools may include any tool for insertion into the body that may be used with a neurostimulation electrode, including (but not limited to): a probe, a pedicle screw, and an implant.

The system may also include a power source for applying power to the neurostimulation electrode. The power source may be (or may connect to) a controller configured to control the neurostimulation electrode. This system may be used with any appropriate neurostimulation electrode, including a monopolar neurostimulation electrode, a bipole pair, a plurality of monopolar electrodes, a plurality of bipole pairs, and a tight bipole network configured to emit an effectively continuous bipole field, as described herein.

In some variations, the accelerometer is a multiple axis accelerometer. As mentioned, the accelerometer may be a durable/reusable accelerometer, or it may be a disposable accelerometer.

The feedback controller may be coupled to, or may include it own, output. As mentioned above, the output may be a visual output (monitor, light, LED, etc.), or an audible output (speaker, etc.), or any other appropriate output. In some variations, the feedback controller is configured to provide feedback to the tool indicating detection of a nerve.

Also described herein are systems for determining if a nerve is nearby an insertable tool. These systems may include: an insertable tool having a first surface comprising a tight bipole network configured to emit an effectively continuous bipole field; an accelerometer to detect muscle movement upon stimulation of a nerve by the tight bipole network; and a feedback controller configured to receive input from the accelerometer and determine activation of a nerve by the neurostimulation electrode.

Methods of using accelerometer-based systems for determining if a nerve is nearby a tool are also described. For example, a method of controlling a tool insertable into a human body may include the steps of: securing an accelerometer to a patient's body; inserting a tool into the patient's body; applying energy to a neurostimulation electrode on the surface of the tool; and monitoring the accelerometer to determine muscle twitch resulting from the application of energy to the neurostimulation electrode. The method may also include the step of comprising providing feedback to the tool based on the output of the accelerometer.

The step of monitoring the accelerometer may also include filtering the output of the accelerometer to remove artifact. Any appropriate filtering may be used, including spectral (power/frequency) filtering, band pass filter, high pass filtering, low pass filtering, and the like. In some variations the accelerometer is ‘tuned’ (e.g., sensate to) a particular range of motion that corresponds to muscle twitch due to nerve stimulation. The step of monitoring the accelerometer may also include the step of synchronizing the monitoring of the accelerometer with the application of energy to the neurostimulation electrode.

The step of applying energy to a neurostimulation electrode may also include applying energy to a tight bipole network to emit an effectively continuous bipole field. Accelerometer-based detection systems may be particularly useful for determining when a nerve is adjacent or in contact with a tool or device including the tight bipole pair networks described.

An accelerometer may be applied to the patient in any appropriate manner, including applying to the surface of the patient's skin. For example, the accelerometer may be adhesively applied, or may be applied using a wrap or strap that secures it to the patient. In some variations a garment is worn that includes one or more integrated accelerometers. The step of applying an accelerometer to the surface of a patient's body may include applying a plurality of accelerometers to the surface of the patient's body. In some variations the accelerometer may be implanted into the patient.

Also described herein are methods of controlling a tool insertable into a human body using the accelerometer-based systems described. For example, a method may include the steps of: securing an accelerometer to a patient's body; inserting a tool into the patient's body; applying energy to a tight bipole network to emit an effectively continuous bipole field on the surface of the tool; and monitoring the accelerometer to determine muscle twitch resulting from the application of energy to the tight bipole network. As mentioned above, the method also includes the step of providing feedback to the tool based on the output of the accelerometer.

### BRIEF DESCRIPTION OF THE DRAWINGS

FIG. 1A shows an example of a generic device including an elongate body and a bipole pair.

FIGS. 1B and 1C show a tight bipole pair.

FIGS. 1D-1F show bipole networks.

FIGS. 2A-2D are various views of portions of a neurostimulation device, according to one embodiment of the present invention.

FIG. 3 is cross-section through a device showing four circumferential regions.

FIG. 4 is another cross-section through a device having four circumferential regions.

FIGS. 5A and 5B illustrate side views and cross-sectional views, respectively, of one variation of a portion of a nerve localization device.

FIGS. 6A and 6B illustrate side views and cross-sectional views, respectively, of another variation of a portion of a nerve localization device.

FIGS. 7A and 7B illustrate side views and cross-sectional views, respectively, of another variation of a portion of a nerve localization device.

FIG. 8 is a side view of a nerve localization device showing multiple current path direction features.

FIG. 9 is a circuit diagram of one variation of a portion of a nerve localization device.

FIG. 10 is a perspective view of a portion of a nerve localization device having two electrodes with rotating brushes.

FIGS. 11A-11C are simplified diagrams of one variation of a nerve localization device.

FIG. 11D is a partial, simplified diagram of a rongeur tip configured as a nerve localization device.

FIGS. 12A-12C illustrate elongate bodies having a plurality of regions each including at least one bipole pair.

FIGS. 13A-13D show partial cross-sections through various devices having elongate bodies including multiple regions.

FIGS. 14A-14B illustrate one variations of a device employed in tissue.

FIG. 14C illustrates another variation of a device in tissue.

FIGS. 14D and 14E show a cross-section and a partial perspective view, respectively, of a device having an elongate body including four regions.

FIG. 14F show a schematic illustration of an electrode that may form part of a tight bipole pair.

FIG. 15 is a cross-section through another variation of a device.

FIGS. 16A-16D illustrate exemplary signals that may be applied to one or more bipole pairs or networks within a region of a device.

FIG. 17A illustrates a system for determining if a nerve is nearby applied to a patient.

FIG. 17B-17D are simplified diagrams of sensors which may be used as part of a system for determining if a nerve is nearby.

FIGS. 18A-18B illustrate variations of a device for determining if a nerve is nearby.

FIGS. 19A-19C are flow diagrams illustrating method of determining if a nerve is nearby a region of a device.

FIG. 20 is a block diagram illustrating components that may be part of a system for determining if a nerve is nearby a device.

FIG. 21 is a cross-sectional view of a spine, showing a top view of a lumbar vertebra, a cross-sectional view of the cauda equina, and two exiting nerve roots.

FIG. 22 is a side view of a lumbar spine.

FIG. 23 is a cross-sectional view of a spine, illustrating a minimally invasive spinal decompression device and method including the use of neural localization as described herein.

FIG. 24 is a block diagram of one variation of a nerve tissue localization system.

FIG. 25 is a perspective view of a nerve tissue localization system.

FIGS. 26A-26F are cross-sectional views of a spine, illustrating one method for using a nerve tissue localization system.

FIGS. 27A-27H are cross-sectional views of a spine, illustrating another method for using a nerve tissue localization system.

FIGS. 28A and 28B show variations of devices for determining if a nerve is nearby.

FIGS. 29A-29C show one variation of a rongeur including a tight bipole network capable of determining if a nerve is in the cutting region of the rongeur.

FIGS. 29D and 29E illustrate other variations of a rongeur including a tight bipole network.

FIG. 30 is a schematic illustrating an accelerometer-based system for determining if a nerve is nearby a neurostimulation electrode.

### DETAILED DESCRIPTION

Described herein are devices, systems and methods for determining if a nerve is nearby a device or a region of a device. In general, a device for determining if a nerve is nearby a device includes an elongate body having an outer surface with one or more bipoles arranged on the outer surface. These bipoles may also be referred to as tight bipoles, and include a cathode and an anode that are spaced relatively close together to form a limited broadcast field. The broadcast field may be referred to as the bipole field, or the field formed by the excitation of the bipole pair. In general, the bipole filed is a controlled or “tight” broadcast field that extends from the bipole pair(s).

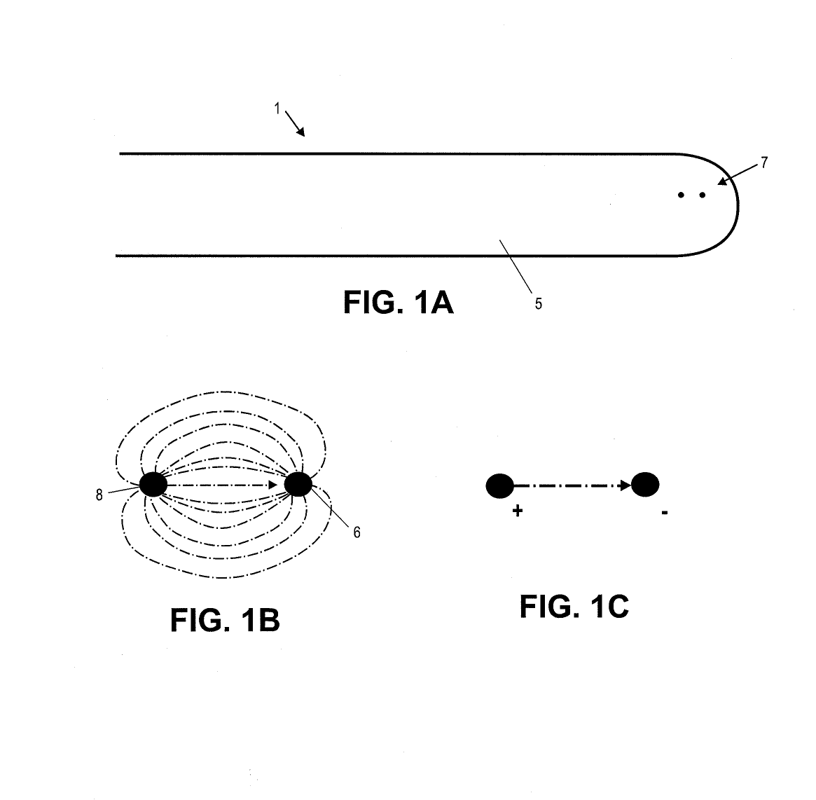

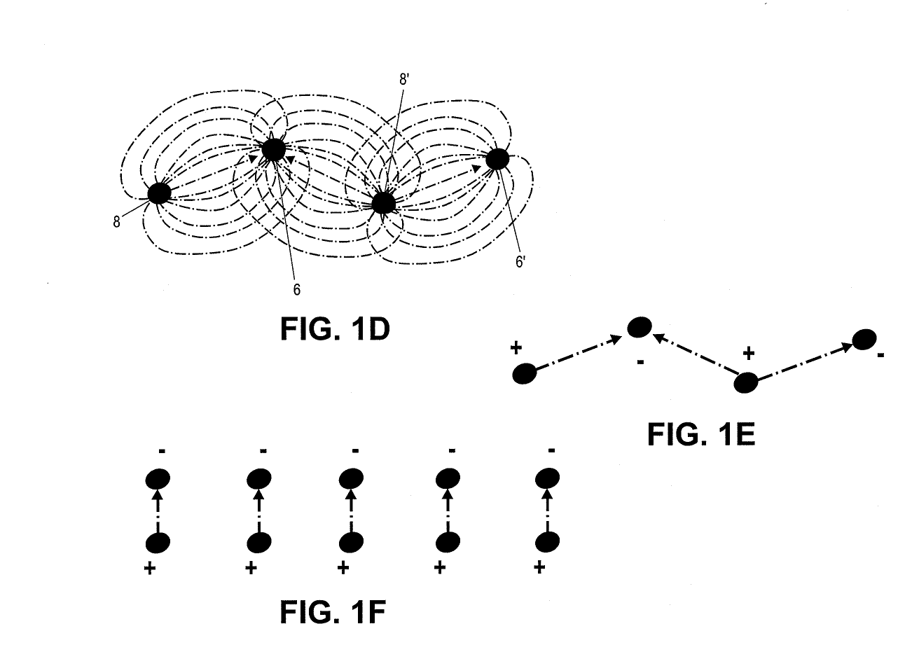

A device for determining if a nerve is nearby the device may be referred to as a nerve localization device, a localization device, or a neurostimulation device. The elongate body region of the device may be referred to as a probe, although it should be understood that any appropriate surgical or medical device may be configured as a device for determining if a nerve is nearby the device. Particular examples of such devices are described below. For example, FIG. 1A shows a generic device 1 configured as a nerve localization device that having an elongate body 5 that may be configured to determine if a nerve is nearby.

The outer surface of a device for determining if a nerve is nearby a region of the device may have two or more regions. In some variations, each region includes two or more bipole pairs that are arranged to detect a nearby nerve. The regions may be arranged around or along the outer surface of the device. For example, the regions may be circumferential regions that divide the outer surface up along the circumference. Examples of different regions are described below. Each region may include one or more bipole pairs, which may be used to detect a nearby nerve.

Returning to FIG. 1A, the elongate body 5 has an outer surface with a blunt (atraumatic) end. In general, the outer body of the device 5 may be formed of any appropriate material, including polymeric materials such as PEBAX, PEEK or the like. Non-conducting and biocompatible materials may be particularly preferred. In FIG. 1A, a single bipole pair 7 is shown near the distal end of the device. FIG. 1B illustrates an approximation of the current lines for a dipole pair, including the cathode 8 and the anode 6. These current lines reflect the dipole field to broadcast field for the dipole pair.

A tight bipole pair may have a very limited broadcast field, as reflected in FIG. 1C, which shows the bipole pair of FIG. 1B having only the major current line. In some variations the size of the anode 6 and cathode 6 forming the bipole pair are relatively small, particularly (e.g., less than 5 mm², less than 3 mm², less than 2 mm², less than 1 mm²), and the anode and cathode are positioned sufficiently nearby so that the majority of current passes between the anodes and cathodes. For example, the anode and cathode of a bipole pair may be separated by less than 5 mm, less than 2 mm, less than 1 mm, etc.

The limited broadcast field may allow stimulation of only nerves that are very near the bipole pair. This may enhance accuracy, and help prevent or limit tissue damage, particularly at the low stimulation.

When a region of the outer surface of a device includes more than one bipole, the bipoles may be arranged as a bipole network. A bipole network includes at least two bipoles that are formed by at least three electrodes (e.g., two anodes and a cathode or two cathodes and an anode). The bipole network is typically arranged so that all of the bipoles in the network are activated synchronously to create an effectively continuous bipole field along the outer surface. For example, FIGS. 1D and 1E illustrates an example of an effectively continuous bipole filed. In this example, the anodes and cathodes forming the bipolar network are arranged so that the current between the two electrodes forms a zigzag pattern. Bipole pairs are located adjacent to each other and share either an anode or a cathode. FIG. 1F illustrates another example of a bipole network, in which adjacent bipole pairs do not share anode or cathodes. This bipole network also forms an effectively continuous bipole field along the outer surface of the device. Adjacent bipole pairs are positioned close to each other.

In some variation all of the cathodes forming a bipole network are electrically connected to each other and all of the anodes forming a bipole network are electrically connected. For example, the anodes of the bipole network may all be formed from a single anodal connector, and all of the cathodes of a bipole network may be formed from a single cathodal connector. Alternatively, all of the cathodes of the bipole network may be formed separately and connected distally on the device. For example, all of the cathodes may be wired to a single connector that connects to a power source or controller configured to energize the bipole network in a particular region.

A device may include multiple bipole networks. For example, different regions on the surface of the device may include different bipole networks (e.g., each region may have its own bipole network). The bipole networks in different regions may be non-overlapping, and may form effectively non-overlapping continuous bipole fields. “Effectively non-overlapping bipole fields” means that the broadcast fields of two or more bipole networks do not substantially overlap. For example, the component of a broadcast field (e.g., intensity) due to a second bipole network is less than 15% (or 10%, or 8% or 5% or 1%) of the component due to a first bipole network at any position near the first bipole network, particularly at the excitation ranges described herein.

A device for determining if a nerve is nearby may also include a controller for controlling the application of energy to the bipoles. In particular, the application of energy to the bipoles may be coordinated as described in the methods sections below, so that the activation of a nerve can be correlated to a particular region of the surface of the device.

In some variations, the bipole or bipole networks are movable with respect to the outer surface of the device. Moving the bipole (e.g., rotating it a around the outer surface) may allow a bipole field (a tight or narrow broadcast field) to be correlated with different regions of the device. This is also described in greater detail below.

### Nerve Localization Devices

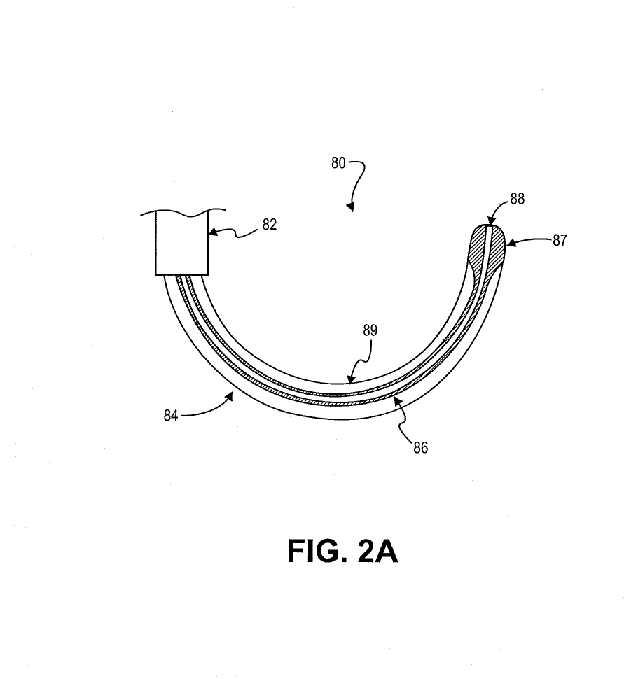

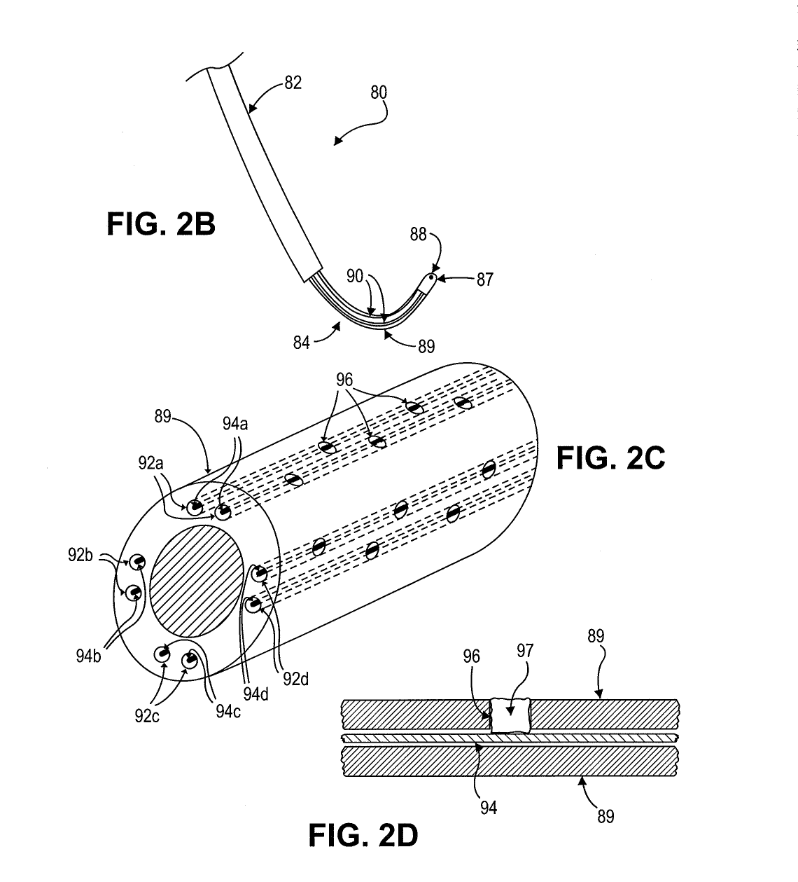

FIG. 2A, illustrates the distal portion of one embodiment of a device capable of determining if a nerve is nearby. This exemplary device 80 is shown in partial cross-section. For clarity, FIG. 2A does not show the bipoles, thus showing more clearly the structure of probe device 80. In this example, the device 80 includes a rigid cannula 82 (or tube or needle) and a curved, flexible guide 84 that can slide through cannula 82. The guide 84 may include a Nitinol core 86 (or inner tube) having a central lumen 88 and an atraumatic, rounded tip 87 and may also include a sheath 89 (or coating or cover) disposed over at least part of Nitinol core 86. The sheath 89 may comprise, in one embodiment, a polymeric material such as PEBAX, PEEK or the like, or any other suitable material, and may form an outer surface having different regions. Core 86 may be made of Nitinol or may alternatively be made of one or more other substances, such as spring stainless steel or other metals. Lumen 88, in some embodiments, may be used to pass a guidewire.

FIG. 2B is a perspective view of a portion of the probe 80 of FIG. 2A, in which two electrically conductive members 90 are visible. One member may be a cathodal conductor and one member may be an anodal conductor. A probe may include as many electrode pairs as desired, such as eight, sixteen, thirty-two, etc. In this example, the probe may have a preformed, curved shape and may be made of at least one flexible, shape memory material, such as Nitinol. In this way, guide 84 may be passed through cannula 82 in a relatively straight configuration and may resume its preformed curved shape upon exiting a distal opening in cannula 82. This curved shape may facilitate passage of guide 74 around a curved anatomical surface, such as through an intervertebral foramen of a spine.

The exemplary device shown in FIGS. 2A-2D may include at least one bipole network, including a plurality of anodes and cathodes. In this example, anodes of a single bipole network are all formed from the same anodal conductor, and the cathodes of the same anodal conductor are all formed from the same cathodal conductor. FIG. 2C illustrates this. In FIG. 2C a section of probe sheath 89, including the outer surface region, is shown in more detail. In one embodiment, sheath 89, which fits directly over at least a portion of Nitinol core 86 (FIG. 2A), includes multiple, longitudinal lumen 92, each of which may contain an electrical conductor 94 forming a plurality of electrodes (e.g., anodes or cathodes). In some embodiments, conductors 94 may be slideably disposed inside lumen 92, while in other embodiments they may be fixedly contained therein. Openings into the sheath 89 form the plurality of cathodes and anodes. The openings may be pores, holes, ports, slits, grooves or the like. Each aperture 96 may extend from an outer surface of sheath 89 to one of conductor lumen 92. As such, apertures 96 may help direct current along paths from one electrical conductor (e.g., cathodal conductor) to the other electrical conductor (e.g., anodal conductor) forming the plurality of bipolar electrode pairs. In some embodiments the conductor 94 may partially extend through and above of the aperture 96 surface. This may be achieved by a conductor 94 that has several bends enabling the apex of the bend to protrude through the aperture 96. Alternatively, the conductor 94 may have sections of its length near the aperture 96 that have a larger diameter than other sections of conductor 94. In a given embodiment, any number of lumen 92, electrical conductors 94 and apertures 96 forming anodes or cathodes may be used. In some embodiments, apertures 96 may extend along a desired length of sheath 89 to approximate, for example, a length of an area to be treated by a device or procedure.

FIG. 2D shows a section of sheath 89 is shown in cross section, showing an electrical conductor 94 comprising (i.e., a cathodal conductor) and a current directing aperture 96 (i.e., forming a cathode of a bipole). In some embodiments, some or all of apertures 96 may be filled with a conductive material 97, such as a conductive gel, solid, matrix or the like. Conductive material 97 may serve the dual purpose of helping conduct electric current along a path and preventing non-conductive substances from clogging apertures 96.

The example shown in FIGS. 2C-2D has four circumferential regions spaced around the circumference of the outer surface of the sheath region of the device. In this example, each region includes a bipole network formed by an anodal and cathodal conductor that is positioned in parallel. Thus, the bipole network (similar to that shown in FIGS. 1D and 1E) extends along the length of each surface region of the device, and may form an effectively continuous bipolar field along the outer surface.

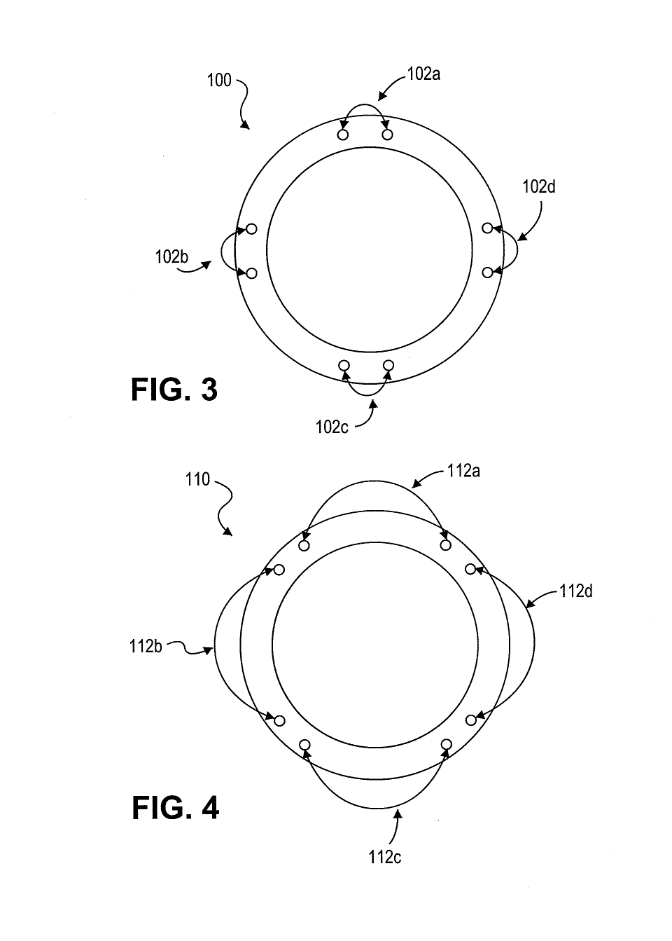

FIG. 3 illustrates a similar arrangement having four regions which each include electrical connectors within the elongate body that may form the bipole network. For example, in FIG. 3, four pairs 102 of anodal and cathodal conductors are shown. The conductors of each pair 102 are close enough together that electric current is transmitted only between electrodes formed by each pair 102a and not, for example, between electrode pairs formed by other anodal or cathodal conductors 102b, 102c, 102d. In some embodiments, the anodal conductor and the cathodal conductor may be “switched” to change the direction that current is passed between electrodes formed by the two conductors. For example, one conductor of each pair 102 may be designated as the transmission conductor (cathode), and the other electrode of the pair 102 may be designated as the return electrode (anode). When one of the conductors forming the anode or cathode is set to ground, this ground may be isolated from the ground (e.g., an anodal conductor) in other regions of the device, which may help isolate the current to the bipolar network in a single region of the device. In various embodiments, electrodes forming the bipole pair may be spaced at any suitable distance apart by spacing the electrical conductors forming the electrodes of the bipole pair. For example, electrodes of each pair may be spaced about 0.1 mm to about 2 mm apart, or about 0.25 mm to about 1.5 mm apart, or about 0.5 mm to about 1.0 mm apart.

FIG. 4 shows another example of a cross-section through a device having pairs 112 of electrical conductors that may form a network of bipole pairs on the surface of the device. In this example, the anodal and cathodal conductors are spaced farther apart. Farther spaced electrode pairs 112 may allow current to pass farther into tissue but may also risk dispersing the current farther and potentially being less accurate. Depending on the specific use and desired characteristics of the device (e.g., sheath 110), the bipole pairs formed may be spaced at any of a number of suitable distances from one another.

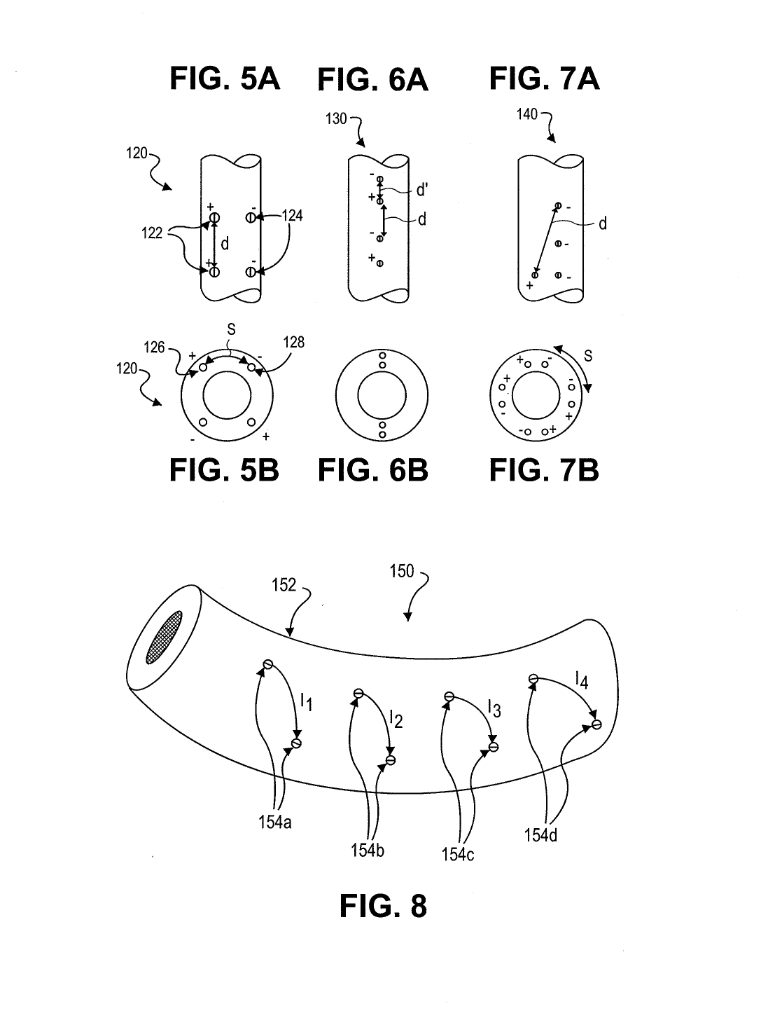

Alternative arrangements of bipole pairs formed from an anodal and cathodal conductor are shown in FIGS. 5A-7B. For example, FIG. 5A is a side-view of a pair of bipole pairs that are formed by apertures 122, 124 in the body of the device (sheath 120) which expose portions of the cathodal electrical conductor 126 and portions of the anodal conductor 128. Apertures forming the cathodes 122 and anodes 124 are disposed along a length of sheath 120 separated by a distance d. As shown in FIG. 5B, the electrical conductors (i.e., cathodal conductor 126 and anodal conductor 128) are embedded in the elongate body and are spaced apart from each other about a circumferential distance s. In one embodiment, the distance d may be greater than the distance s, so that current is more likely to travel circumferentially between positive and negative electrodes, rather than longitudinally along sheath 120. As can be appreciated from FIGS. 6A and 7A, current may be directed along any of a number of different paths in different embodiments of elongate body (sheath 120), by changing the separation distances of apertures 122, 124 providing access to the electrical conductors 126, 128.

For example, in FIGS. 6A and 6B, the cathodal and anodal conductors are positioned in immediately above and below one another, and apertures forming the anodes and cathodes of bipole pairs may be spaced at different distances along the body of the device 130, such that current is more likely to travel between two closer spaced apertures (distance d′) than between two farther spaced apertures (distance d).

In FIGS. 7A and 7B, current may be directed along a distance d between apertures forming anodes and cathodes of bipole pairs that are spaced more closely together than the anodal and cathodal conductors of other bipole pairs. As mentioned above, in various embodiments of these nerve localization devices, any combination of anodal or cathodal conductors, apertures forming the anode and cathode pairs, and/or other current direction path features may be included.

FIG. 8 shows a portion of a nerve localization device 150. This nerve localization device variant includes a sheath 152 having multiple current directing apertures 154 disposed over a cathodal conductor and an anodal conductor, forming bipole pairs along the outer surface of the device. As shown, current may be driven along multiple paths between pairs of apertures 154a, 154b, 154c, 154d. Multiple individual currents I1, I2, I3 and I4 add up to the total current IT transmitted between the anodal and cathodal conductor. In various embodiments, the bipole pairs formed 154 may be disposed along any desired length of probe 150. Any number of bipole pairs may be included. As mentioned above, in some variations the cathodes and/or anodes formed in a single region of the device may be formed from multiple (including individual) anodal/cathodal conductors (e.g., wires).

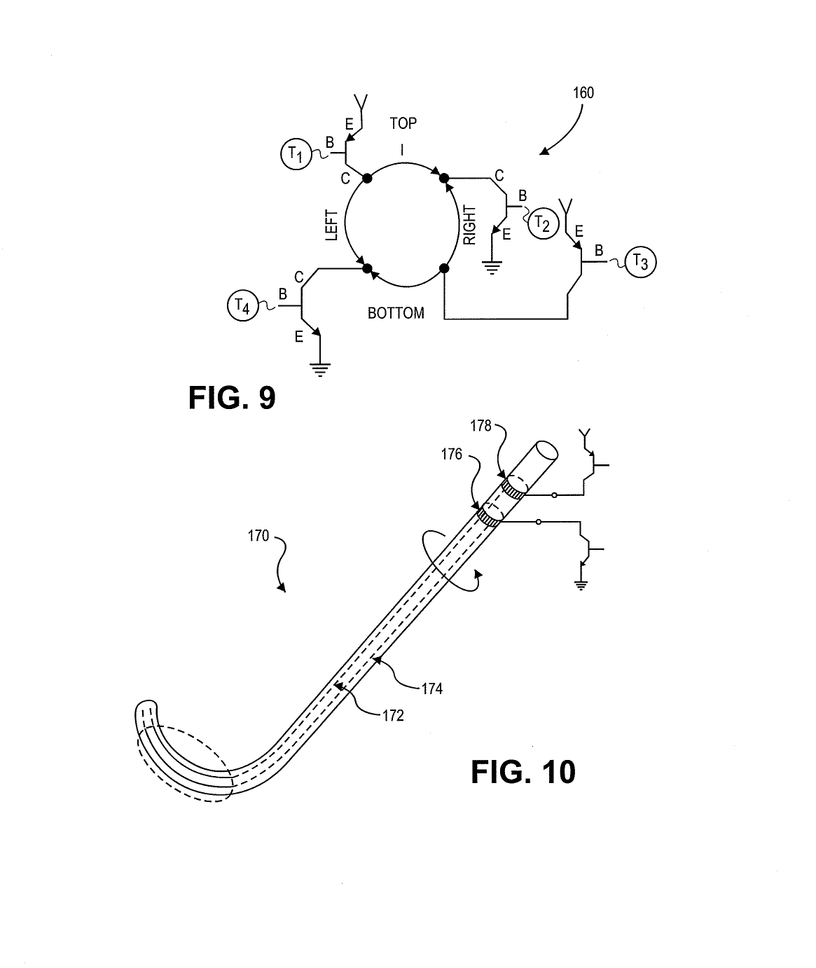

FIG. 9 is a circuit diagram 160 for a nerve localization device having two bipole pairs (e.g., eight electrical conductors). In this simple form, electric current may be driven between the electrical conductors along a top, bottom, left and right side, separately. Each of these side forms a different region of the device.

Another example of a nerve localization device is shown in FIG. 10. In FIG. 10, the nerve localization device includes two electrical conductors 172, 174 forming at least one bipole pair (not shown) and two rotating brushes 176, 178. Such an embodiment may allow different sides, such as top, bottom, left and/or right sides, to be stimulated with only two electrodes 172, 174, rather than multiple electrode pairs in different sections.

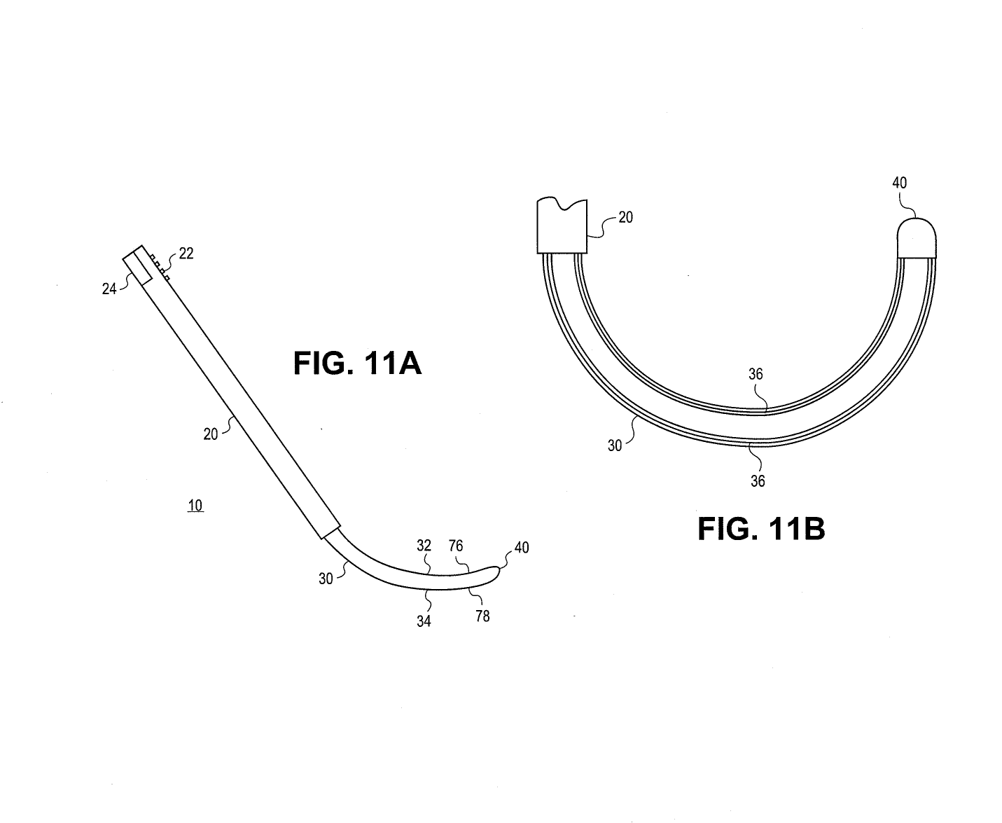

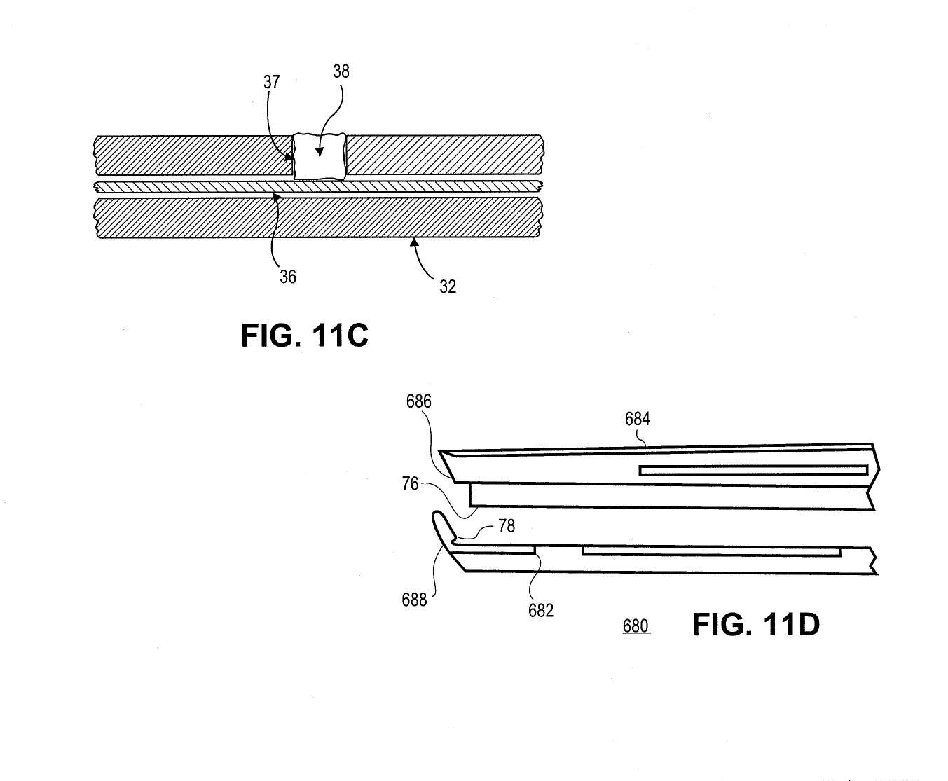

The elongate bodies forming part of the nerve localization devices described above may be used with any appropriate controller and/or stimulator configured to energize the bipole pairs. Thus, any of these devices may be used as part of a system including a controller and/or stimulator. In some variations, the elongate body may also be referred to as a probe. Examples of elongate bodies, including elongate bodies having different regions which may each contain one or more bipole pairs, are shown in FIGS. 11A-13D.

FIG. 11A is a simplified diagram of one variation of a device 10. This device 10 may be used to perform one or more medical procedures when orientation of the device with respect to an adjacent nerve is desired. Similar to the device shown in FIG. 2A above, this variation 10 includes a cannula 20 and a probe 30. The device 30 includes a tip 40, a top section 32, and a bottom section 34. The device 30 may include multiple bipole pairs 76, 78 or bipole networks consisting of multiple bipole pairs. A first bipole pair or bipole network 76 may be located on a first section 32 and a second bipole pair 78 may be located on a second section 34. In one variation the bipole network or pair 76 may be energized to determine whether a nerve is located near or adjacent to the first or top section 32. The second bipole network or pair 78 may be energized to determine whether a nerve is located near or adjacent to the second or bottom section 34. The first bipole network or pair 76 and the second bipole network or pair 78 may be alternatively energized to independently determine whether a nerve is located near or adjacent to the first section 32 and/or the second section 34.

In some variations a bipole pair or network 76, 78 is typically energized with one or more electrical signal(s). The device may monitor the electrical signal applied to the bipole network (or pair) 76, 78, and may monitor the characteristics of the electrical signal and determine whether tissue is near or adjacent the bipole(s) 76, 78 as a function of the monitored electrical signal characteristics. The electrical signal characteristics may include amplitude, phase, impedance, capacitance, and inductance over time or frequency.

After an electrical signal is applied to the bipole network or pair 76, 78, an output may be detected. In some variations the nerve localization device includes a sensor or sensors for monitoring the nerve response. For example, the device may monitor one or more sensors anatomically coupled to nerve or afferent tissue enervated by the nerve whose condition is modified by the signal(s) applied to the bipolar network or pair 76, 78. For example, the device may monitor one or more sensors innervated by the nerve tissue such as limb muscles.

The nerve localization devices and systems described herein may include one or more indicators or outputs 22, 24. The detectors may provide a user-identifiable signal to indicate the location of the nerve or the status of the system. For example, the nerve localization devices may include one or more light emitting diodes (LEDs), buzzers (or other sound output), a video display, or the like. An LED may be illuminated based on signals generated by, received by, or generated in response to the energized bipole(s) 76 or 78 as discussed above. In some variations the system or devices create a vibration or sound that a user manipulating the device 20 may feel or hear. The intensity of the output may vary as a function of detected signal.

As shown in FIG. 11B, a nerve localization device may include a pair of electrical conductors 36 (anodal conductor and cathodal conductor) which form one or more bipole pairs. The anode or a cathode of the bipole pair(s) 76, 78 may be formed as described above via an opening 37 filled with a conductive material 38, such as a conductive gel, solid, matrix, or other conductive material. An example of this is shown in FIG. 11C. Alternatively, the bipole pair 36 and the conductive material 38 could be formed from the same conductive elastic or semi-elastic material. The elongate body of the device 30 may include a bipole network comprising bipole pairs that are configured in a coil or zig-zag pattern along the length of the probe. This arrangement may help ensure continuous conduction during flexion of the probe 30. In another variation, the anodal and/or cathodal conductors are formed of conductive ink (e.g., loaded in an elastomeric matrix) may be deposited on the outside of the probe. The conductive ink could be insulated with the exception of discrete points forming the anode or cathode of the bipole pair. In another embodiment a thin flex circuit could be wrapped around probe to construct the bipoles.

FIG. 11D is a partial, simplified diagram of one variation of a rongeur jaw 680 configured as a nerve localization device. In this variation the rongeur jaw forms the elongate body of the device on which at least one bipole pair is located. The rongeur jaw 680 may include a lower jaw 682 and an upper jaw 684. The lower jaw 682 may have a tip 688 and a bipolar network or pair 78 on an inner surface. The upper jaw 684 may have a tip 686 and a bipolar network or pair 76 on an inner surface. In one variation, the first bipolar network or pair 78 may be energized to determine whether a nerve is located near or adjacent to the first or bottom jaw 682. The second bipole network or pair 76 may be energized to determine whether a nerve is located near or adjacent to the second or top jaw 684. The first bipolar network or pair 76 and the second bipolar network or pair 78 may be alternatively energized to independently determine whether a nerve is located near or adjacent to the first, bottom jaw 682 and/or the second, upper jaw 684.

In operation, a user may employ such a device to ensure that a nerve is located between the lower jaw 682 and upper jaw 684 or that a nerve is not located between the lower jaw 682 and upper jaw 684. A user may then engage the rongeur jaws 680 to excise tissue located between the jaws 682, 684. A user may continue to energize or alternately energize the bipole networks or pairs 76, 78 on either jaw while excising tissue.

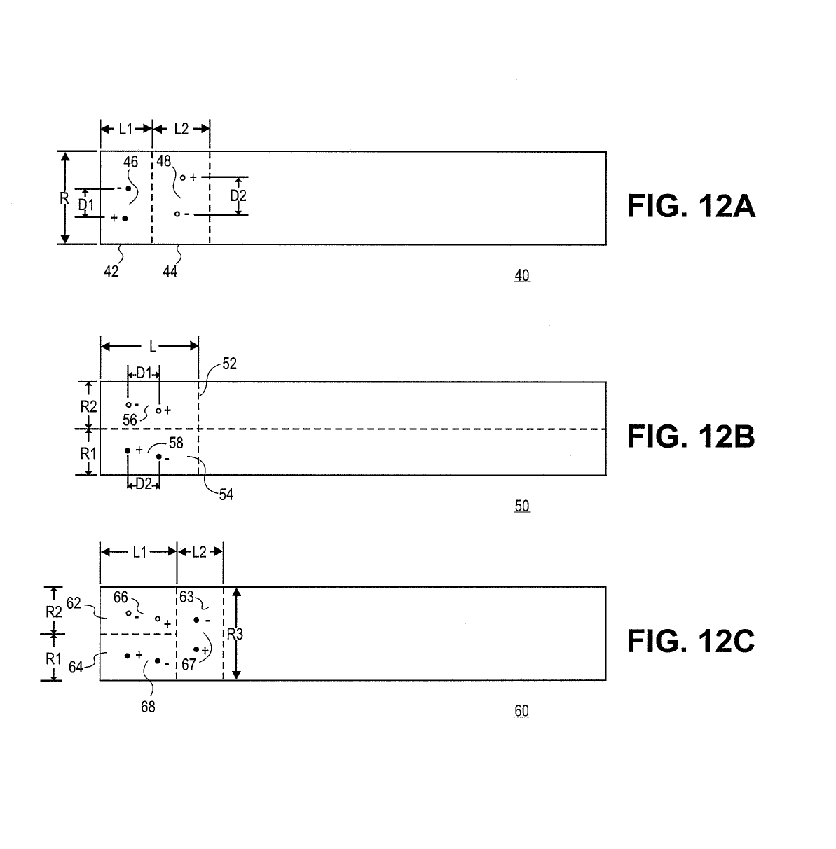

FIGS. 12A-12C are examples of elongate bodies having regions which include at least one bipole pair, and may include a bipole network. Each elongate body in FIGS. 12A-12C (40, 50, and 60, respectively) may be part of a device or system capable of determining if a nerve is nearby the device, and may be configured as part of surgical instrument such as a rongeur 680, or other instrument. The configuration 40 shown in FIG. 12A includes two longitudinal regions 42, 44 at the distal end. The distal section 42 has a longitudinal length L1 and a width R, which may also be referred to as a radial length. The more proximal section 44 has a longitudinal length L2 and a width of R. Each region 42, 44 includes at least one bipole pair 46, 48. A bipole pair 46, 48 typically includes at least one anode (−) and cathode (+) that can be excited to create a restricted current pathway between the anode and cathode 46, 48.

The distance between the anode and cathode pair of may be less than the distance between any of the electrodes forming part of a bipole pair in an adjacent region of the elongate body. For example, the electrodes forming the bipole pair (or bipole network) in the first region 42 are closer to each other than to either the anode or the cathode in the adjacent region 44. Likewise, the distance between the anode and cathode pair in the second region 44 is less than the distance between the anode and the cathode of the first region. For example, the distance between the anode and cathode forming bipole pairs in the first region 42 is labeled D1 and the distance between the anode and cathode in the bipole pair in the second region is labeled D2. D1 may be less than or equal to L1 and R and D2 may be less than or equal to L2 and R. Any appropriate spacing (D1 or D2) may be used between the anodes and cathodes forming the bipole pairs. For example, D1 and D2 may be about 0.25 mm to 2.0 mm apart. In one variation D1 and/or D2 are about 0.50 mm. When a bipole or bipole network in a region 46, 48, is energized, current may flow between the anode and cathode along a conductive pathway substantially only within its respective sections 42, 44. This current flow (and/or the related magnetic field) may be referred to as the ‘broadcast field of the bipole pair or bipolar network. A device including regions having tight bipoles or bipole networks 40 may be employed to determine whether a nerve is closer to the first region 42 or the second 44, as described above. The bipole pairs (or bipole networks) in each region may be alternatively energized and an external sensor(s) can be used to monitor and/or determine whether a nerve is closer to the first region 42 or second region 44.

The arrangement of the bipole pairs or bipole network may help determine the sensitivity of the device. For example, D1 may be less than D2, resulting in the bipole pair in the first region having a smaller broadcast field (and a shorter conductive pathway) than the bipole pair 48 in the second region. This may allow detection of a nerve located further from second region than the first region, assuming a nearly equivalent energy is applied to the bipole pairs (or networks) within each region. Of course, the energy applied may be varied between different regions.

FIG. 12B shows an example of an elongate member 50 having two regions 52, 54 separated along the longitudinal (or circumferential if the member is rounded) axis of the member 50. Each region 52, 54 may include one or more a bipole pairs 56, 58. For example, each region may include a bipole network formed of multiple bipole pairs. The individual bipole pairs may share anodes and cathodes, as described above. In this example, the width of the first region is the circumferential or linear distance, R1, and the length is the distance L. The width of the second region is R2 and the length is L. The bipole pairs 56, 58 in each region may be longitudinally oriented, radially oriented, or some combination. For example, a bipole network may have anodes and cathodes arranged in a linear pattern (e.g., extending longitudinally) or a zigzag pattern (also extending generally lineally). Other arrangements are possible.

FIG. 12C shows another variation of an elongate member having three regions, two arranged longitudinally 62, 64, and one more proximally 63, adjacent to the two distal longitudinal (or circumferential) regions. Each region 62, 63, 64 may include one or more bipoles 66, 67, 68 or bipole networks. The spacing between the electrodes forming the bipoles of a bipole pair or network in one of the regions may be less than the spacing to electrodes outside of the region. This may prevent current from passing from an electrode (e.g., anode, cathode) in one region and electrodes in another region. In some variations the controller or device is configured so that the anodes and/or cathodes are electrically isolated (e.g., do not share a common ground) and may be configured to electrically float when not being energized.

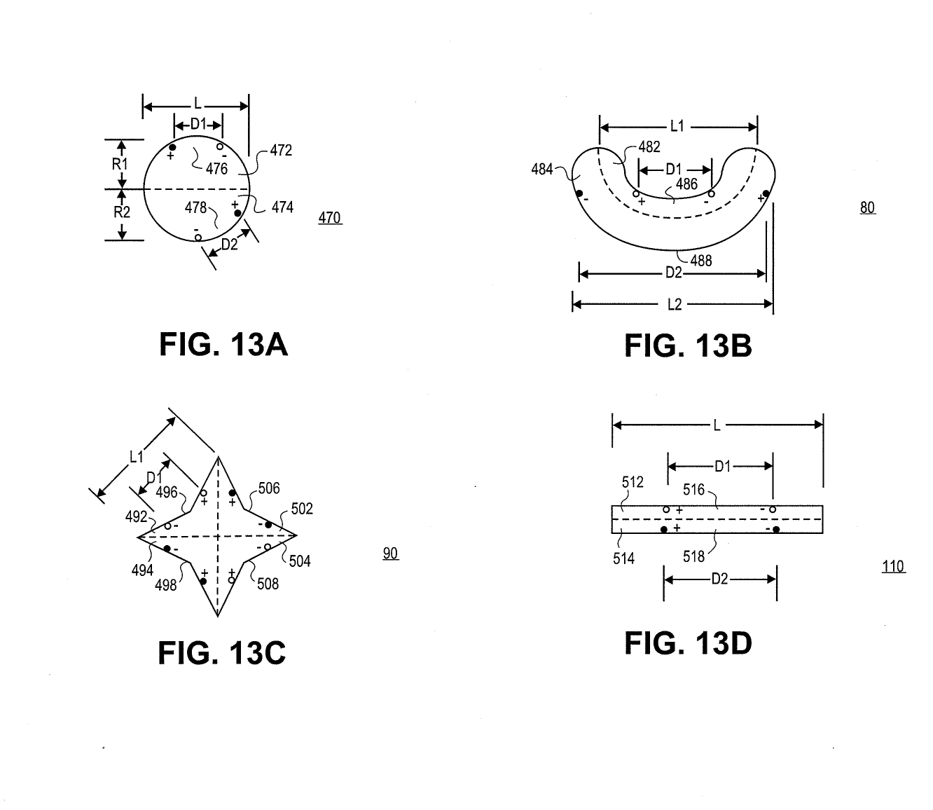

FIGS. 13A-13D show partial cross-sections through elongate members 470, 480, 490, 510 which may be used as part of a device for determining if a nerve is nearby. Each region includes multiple (e.g., two or more) regions that each include one or more bipole pairs (e.g., bipole networks). These examples each have a different cross-sectional shape, and have circumferential regions that are oriented differently around the perimeter of the elongate member. For example, FIG. 13A shows a portion of a device having an outer surface that includes two regions or sections 472, 474 that are circumferentially distributed. Each region 472, 474 includes one or more bipoles 476, 478, having at least one anode (−) and one cathode (+) that can be powered so that current flows between the anode and cathode, resulting in a broadcast field. In this embodiment, the distances between the anode and cathode pairs forming the bipoles in each region are less than the distance between the anode of one region and the cathode of the other region. Region 472 may have a radial length R1 and circumferential span of L (e.g., a width of R1*pi); the longitudinal distance or length is not apparent from this cross-section, but may extend for some distance. In this example, a bipole pair in the first region may have an anode and cathode 476 that are separated by a distance (approximately D1) that is less than half the length of the first circumferential region, and the spacing of the tight bipole pair (approximately D2) in the second region may be less than half the length of the second circumferential region. In one variation, D1 and/or D2 may be about 0.50 mm. In some variations the spacing between the bipole pairs in different regions (and within the same region for bipole networks) is approximately the same.

The configuration 480 shown in FIG. 13B may also include two circumferential regions 482, 484 on the distal end of the elongate member. Each region 482, 484 may include a bipole pair or network 86, 88, as described above. In this embodiment, the distances between the anode and cathode pairs of either of region 486 and 488 are less than the distance between the anode of one region and the cathode of the other region.

The configuration 490 shown in FIG. 13C includes four radial regions 492, 494, 502, 504 which may also each have one or more bipole 496, 498, 506, 508. FIG. 13D has two circumferential regions 512, 514. Each radial region 512, 514 includes at least one bipole pair 516, 518.

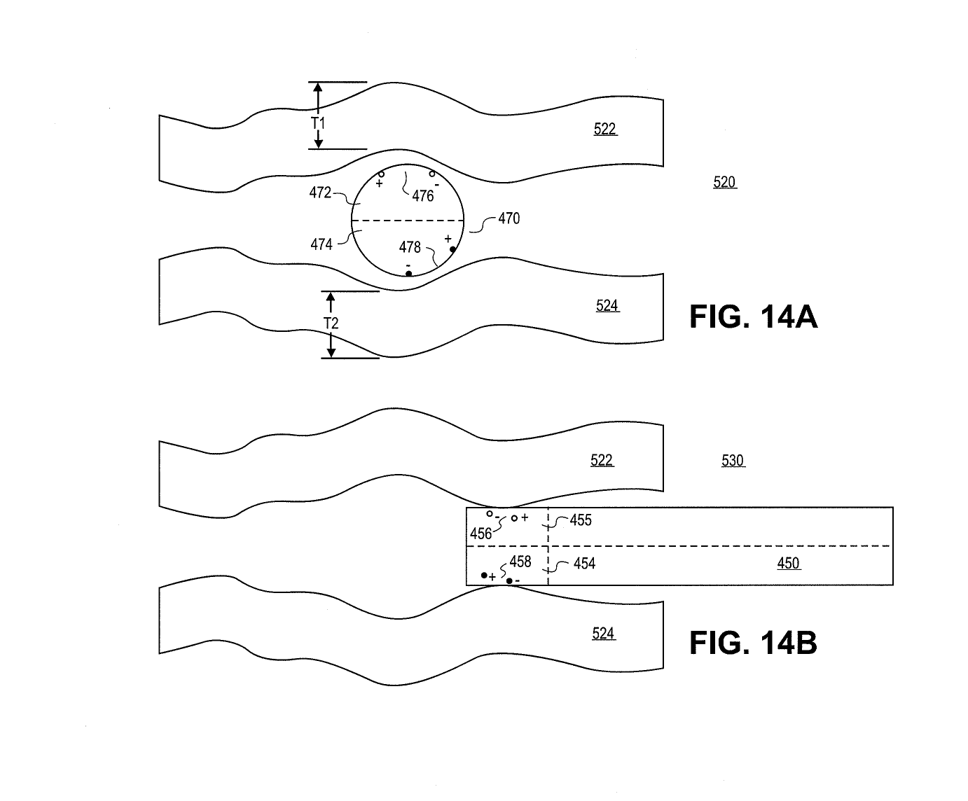

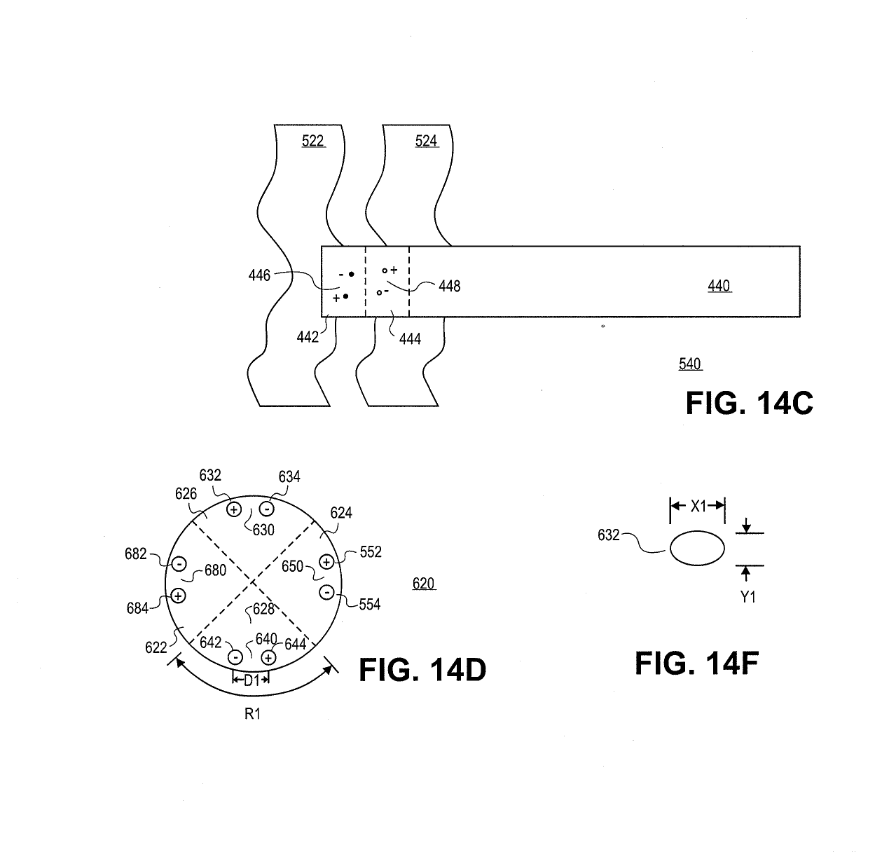

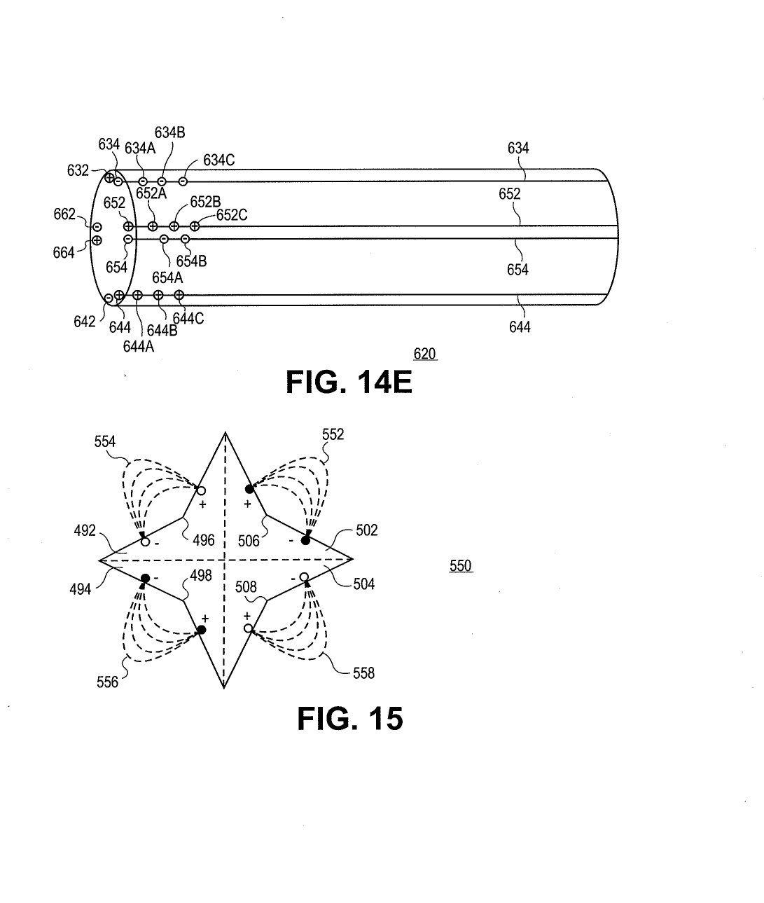

FIGS. 14A-14C are partial diagrams of a portion of a device capable of determining if a nerve is nearby. The device includes an elongate body (shown in cross-section) having to regions with at least one bipole pair in each region. The device is deployed in tissue 522, 524. The device 470 shown in FIG. 14A includes two radially separated regions 472, 474, similar to the device shown in FIG. 13A. Each region 472, 474 has a bipole network or at least one bipole pair 476, 478 having an anode (−) and cathode (+). The device may determine whether the module 476 is near or adjacent a nerve (e.g., in the tissue 522 or 524) as a function of signals generated in response to one or more energized bipole pairs in the regions, as described above. When a bipole pair or network 476 is energized, the conductive pathway (or bipole field) typically does not extend substantially into the tissue 524, 522.

The first region 472 may have a radial length R1 and longitudinal length, L, and the second region 474 may have a radial length R2 and longitudinal length, L. An anode and a cathode forming at least one bipole pair within the first region 472 may be separated by a distance, D1, and an anode and cathode in the second region may be separated by a distance D2. In some variations the energy applied to a bipole pair or network does not project very far into the tissue. This may be a function of the configuration of the bipole pair (e.g., the size and spacing) and the energy applied. For example, the energy projecting in to the tissue from a bipole pair in the first region 472 may not extend substantially further than a distance of T1, so that it would not provoke a response from a neuron located further than T1 from the electrodes. Similarly, the energy projecting into the tissue from a bipole pair (or the bipole network) in the second region 474 may not extend substantially further than a distance of T2 from the electrodes. The electrodes of the bipole pair or network in the first region 472 may be are separated by a distance, D1 that is less than or equal to R1, T1, and L, and the bipole pair or network in the second region 474 may be separated by a distance D2 that is less than or equal to R2, T2, and L. For example, D1 and D2 may be about 0.25 mm to 2.0 mm apart (e.g., 0.50 mm). The energy applied to the bipole pair or network may be limited to limit the projection of energy into the tissue. For example, the current between the bipole pairs may be between about 0.1 mA to 10 mA.

The device may be used to determine if a nerve is near one or more regions of the outer surface of the device, and/or which region the nerve is closest to. For example, a first electrical signal may be applied to the bipole pair/network in the first region 472 for a first predetermined time interval, and a response (or lack of response) determined. A response may be determined by using one or more sensors, it may be determined by observing the subject (e.g., for muscle twitch), or the like. Thereafter a second electrical signal may be applied to the bipole pair/network in the second region 474 for a second predetermined time interval, and a response (or lack of a response) determined. The first predetermined time interval and the second predetermined time interval may not substantially overlap, allowing temporal distinction between the responses to different regions. The device may include more than two regions, and the bipole network may be of any appropriate size or length.

Based on the monitored response generated after the application of energy during the predetermined time intervals, it may be determined if a nerve is nearby one or the regions of the device, or which region is closest. For example, if application of energy to the bipole pairs/networks in both regions results in a response, the magnitude of the response may be used to determine which region is closest. The durations of the predetermined time intervals may be the same, or they may be different. For example, the duration of the first predetermined time interval may be longer than the duration of the second predetermined time interval. The average magnitude of the electrical signals applied may be the same, or they may be different. For example, the magnitude of the signal applied to the bipole pair/network in the first region may be greater than the average magnitude of the signal applied to the second region.

The device 450 shown in FIGS. 14A and 14B includes two longitudinally separated sections 452, 454. Each section 452, 454 has a bipole pair or bipole network 456, 458 that has at least one anode (−) and one cathode (+).

The device 440 shown in FIG. 14C includes two longitudinally separated regions 442, 444, each including a bipole pair or network 446, 448 including at least one anode (−) and one cathode (+). When the bipole pair or network in a region is energized, the device may be used to determine if a nerve is nearby based on the generated response to the energized bipole pair/network.

FIG. 14D shows a cross-section through a region of an elongate body of a device having four regions which each include bipole pairs or networks. The electrodes forming the bipole pairs or networks are connected to an electrically conductive element so that the anode(s) and cathode(s) in a particularly region are all in electrical communication. For example, as illustrated in FIG. 14D, four cathodal conductors 644, 664, 632, 652 pass through the body of the device and electrically connect to electrode regions (not visible in FIG. 14D) on the surface of the device. Similarly, four anodal conductors 642, 662, 634, 654 pass through the body of the device and electrically connect to electrode regions (not visible in FIG. 14D) on the surface. This forms bipole pairs 640, 660, 630, 650. When the cathodal and/or anodal conductors form multiple electrode regions (electrodes) in each region, they may form a bipole network 640, 660, 630, 650.

FIG. 14E is a partial isometric diagram of a device shown in FIG. 14D, in which each region includes a bipole network formed along the lengths of the device. Each bipole network includes anodes formed from a single anodal conductor and cathodes formed from a single cathodal conductor. FIG. 14F is an exemplary illustration of an anode or cathode 632. The anode may have any appropriate shape (e.g., round, oval, square, rectangular, etc.), and any appropriate surface area (e.g., less than 10 mm², less than 5 mm², less than 3 mm², less than 2 mm², less than 1 mm²). For example, in some variations, the height of the anode or cathode (e.g., Y1) may be about 0.25 mm to 0.75 mm, and the width of the anode or cathode (e.g., X1) is about 3× the height (e.g., X1=3*Y1). As mentioned previously, the electrode may be formed of a conductive material (e.g., metal, polymer, etc.), and may be formed by forming a passage into the body of the elongate member until contacting the conductive member, then filling the passage with an electrically conductive material.

The conductive element may be a conductive wire, gel, liquid, etc. that may communicate energy to the anodes or cathodes.

The elongate body may be any appropriate dimension, and may be typically fairly small in cross-sectional area, to minimize the damage to tissue. For example, the outer diameter of elongate member may be about 1.5 mm to 5 mm (e.g., about 2 mm).

FIG. 15 illustrates conductive pathways 550 of one example of a device 490 (similar to the variation shown in FIG. 13C) that includes four radial regions 492, 494, 502, 504 near the distal region of the elongate body. Each bipole pair or network 496, 498, 506, 508 includes at least one anode (−) and cathode (+) that, when energized, creates a limited conductive pathway between the respective anode(s) and cathode(s) of the bipole or bipole network 496, 498, 506, 508. For example, the current pathways 554, 556, 552, and 558 between the bipoles may broadcast energy about 3 to 5 times the distance between the respective cathodes and anodes forming the bipole(s). Thus, the current pathways 554, 556, 558, 552 may be substantially confined to the respective regions 492, 494, 502, 504 of the elongate body forming the bipole or bipole network.

In operation, each bipole network is stimulated separately for a predetermined time. For example, one bipole network 496, 498, 506, or 508 may be energized with a first signal for a predetermined first time interval. Thereafter, another bipole network 496, 498, 506, or 508 may be energized with a second signal for a predetermined second time interval. Different energy levels may be applied, for example, as a function of the tissue 522, 524 that a user is attempting to locate or identify.

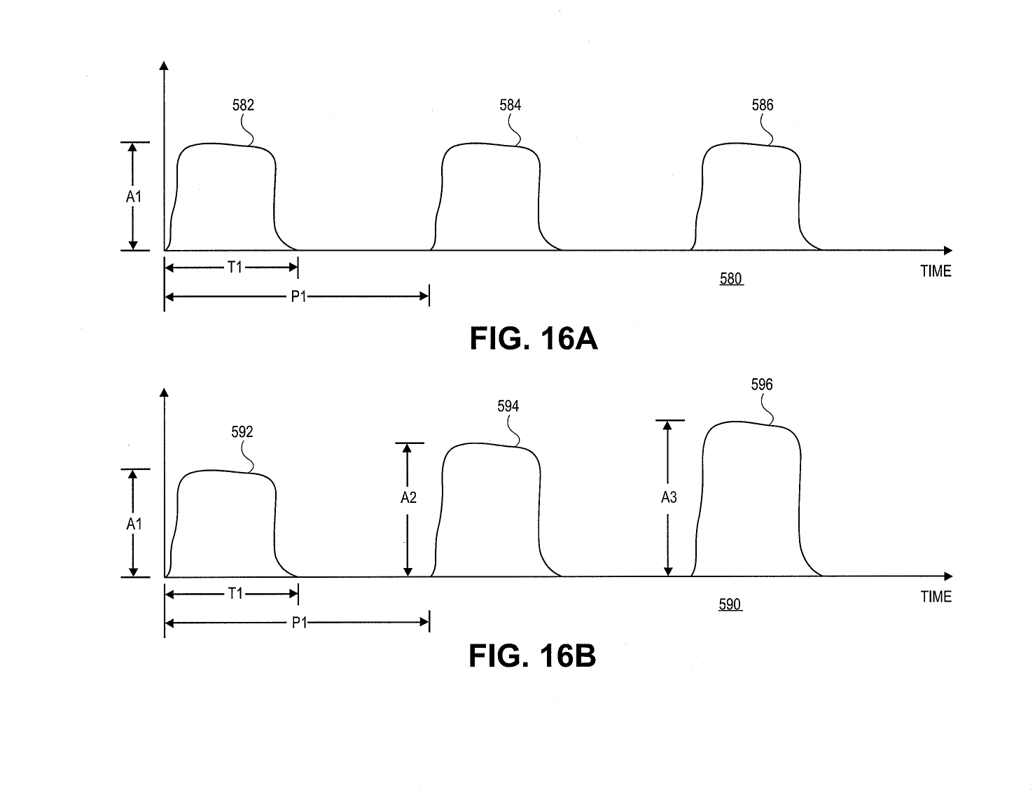

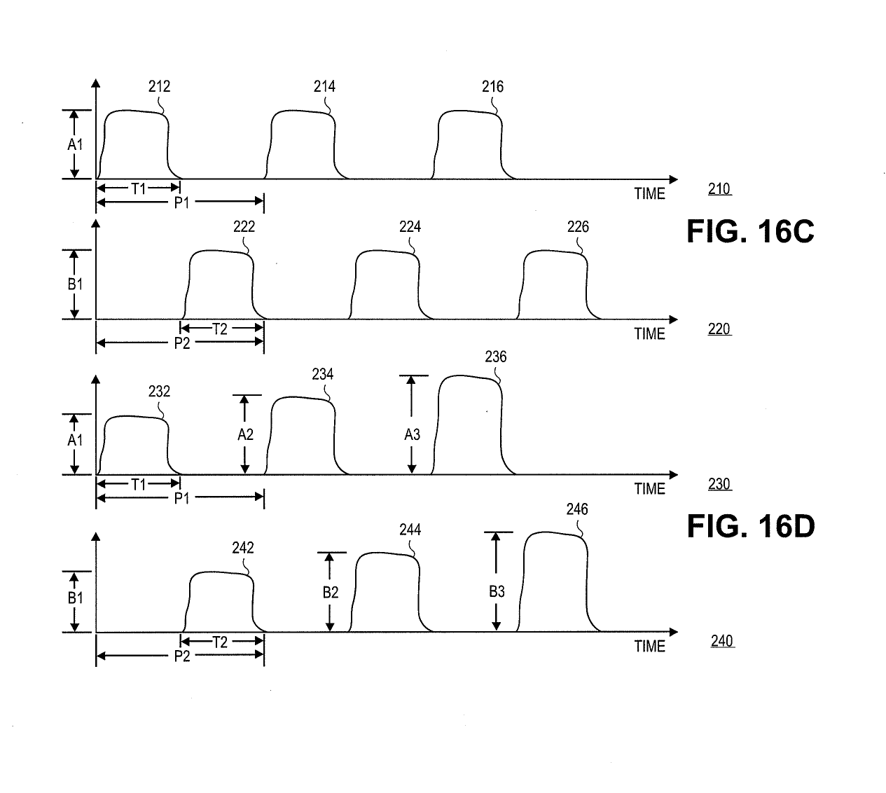

FIGS. 16A-16D are diagrams of electrical signal waveforms 580, 590, 210, 220, 230, 240 that may be applied to one or more bipole pairs (or bipole networks). Exemplary signal waveforms include square-wave pulses 582, 584, 586. Each pulse 582, 584, 586 may a have a similar magnitude and envelope. The square-wave pulses may be idealized (e.g., with square edges, etc.), or rounded (as shown in FIGS. 16A-16D). The waveforms may be used to energize the bipole network periodically P1 for a predetermined interval T1 where each pulse 582, 584, 586 has an amplitude A1. For example, A1 may be about 0.1 milliamperes (mA) to 10 mA, the pulse width T1 may be about 100 microseconds (μs) to 500 μs and the period P1 may from 100 ms to 500 ms. For example, A1 may be about 0.5 milliamperes (mA) to 5 mA, the pulse width T1 may be about 200 microsecond (μs) and the period P1 may about 250 ms as a function of the energy required to depolarize neutral tissue. The applied energy may also be expressed as a voltage.

FIG. 16B illustrates another variation, in which the applied signal waveform 590 includes square-wave pulses 592, 594, 596 that have an increasing magnitude but similar pulse width T1. The waveform 590 may be used to energize a bipole network periodically P1 for a predetermined interval T1 where pulses 592, 594, 596 have increasing or ramping amplitudes A1, A2, A3. The waveform 590 may continue to increase pulse amplitudes in order to identify a nerve (up to some predetermined limit). For example, stimulation of one or more bipole pairs may cycle a ramping stimulation. In one example, A1, A2, and A3 are about 1 milliamps (mA) to 5 mA where A3>A2>A1, the pulse width T1 may be about 100 microsecond (μs) to 500 and the period P1 may from 100 ms to 500 ms. For example, the pulse width T1 may be about 200 microseconds (μs) and the period P1 may about 250 ms.

In FIG. 16C the signals applied to energize different regions of the device are different. For example, a first waveform 210 may be applied to a first bipole network of a device, and a second waveform 220 may be applied to energize a second bipole network of the device. In this example, the signals are interleaved. The signal waveform 210 includes several square-wave pulses 212, 214, and 216 and the signal waveform 220 includes several square-wave pulses 222, 224, and 226. Each pulse 212, 214, 216, 222, 224, 226 may a have a similar magnitude and envelope. The waveform 210 may be used to energize the first bipole network periodically P1 for a predetermined interval T1, where each pulse 212, 214, 216 has an amplitude A1. The second waveform 220 may be used to energize a second bipole network periodically P2 for a predetermined interval T2 where each pulse 222, 224, 226 has an amplitude B1. In some variations, the pulse width T1, T2 is about 100 microseconds (μs) to 500 μs, and the period P1, P2 is from 100 ms to 500 ms. For example, A1, A2 may be about 0.5 milliamperes (mA) to 5 mA, the pulse width T1, T2 may be about 200 microsecond (μs) and the period P1, P2 may about 250 ms. The pulses 212, 214, 216 do not substantially overlap the pulses 222, 224, 226. In some variations, T1>T2 and P2 is an integer multiple of P1.

FIG. 16D is another example, in which different regions of the device are energized with pulses having increasing amplitudes. In this example, an amplitude increasing or ramping pulse waveform 230 may be applied to a first bipole network, and a second amplitude increasing or ramping pulse waveform 240 may be applied to a second bipole network. The signal waveform 230 includes several amplitude increasing or ramping square-wave pulses 232, 234, and 236 and the signal waveform 240 includes several amplitude increasing or ramping square-wave pulses 242, 244, and 246. In variations having more than two regions, each region may be stimulated separately, so that the time period between stimulations (P1-T1) may be larger than illustrated here. Methods may also include changing the stimulation applied, or scaling it based on a response, as described in more detail below.

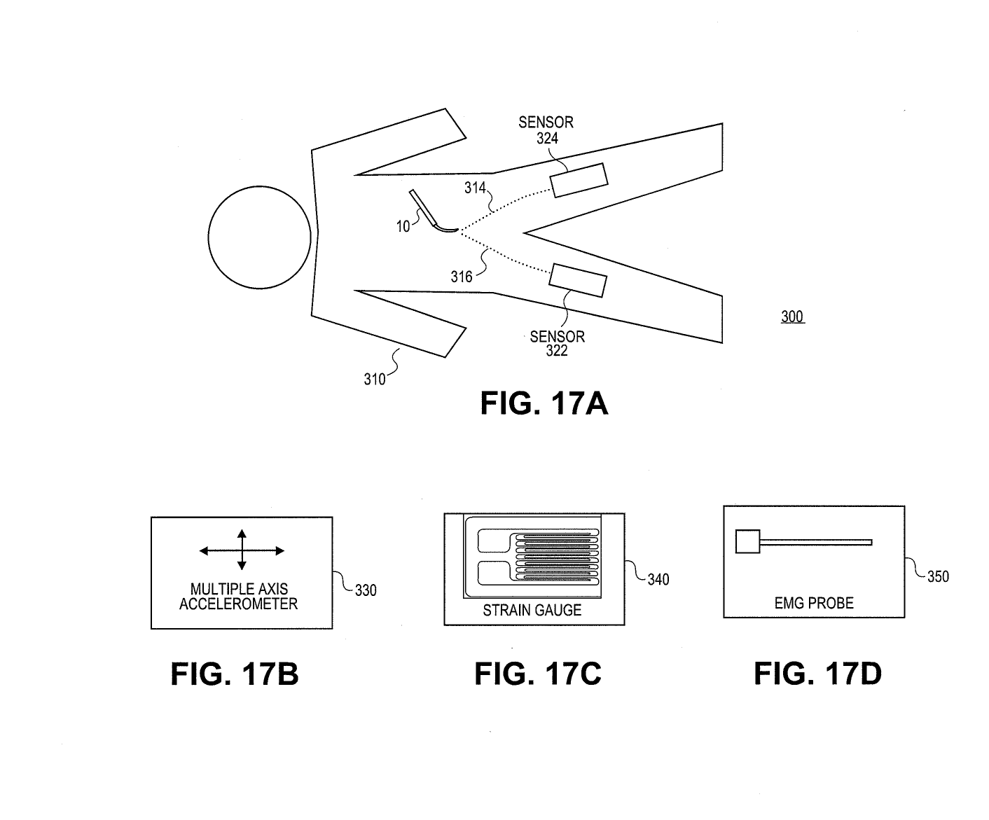

FIG. 17A is illustrates a schematic of a subject 310 in which the device for determining if a nerve is nearby is being used. In this illustration 300, a tissue localization device 10 is used as part of a system including sensors 322, 324. In this system, the device 10 may energize one or more bipole pairs or bipole networks to depolarize neutral tissue that is near a region of the device including the bipole pair or network. A sensor 322 may be placed on, near, or within muscle that may be innervated when neutral tissue is depolarized by a nearby energized bipolar or optical module. The sensor 322 may be innervately coupled to nerve tissue via a neural pathway 316 and sensor 324 may be innervately coupled to nerve tissue via a neural pathway 314. For example, the device may be used as part of a spinal procedure and the sensors 322 may detect an Electromyography (EMG) evoked potentials communicated in part by a patient's cauda equina along the pathways 314, 316.

FIGS. 17B-11D are simplified diagrams of sensors 330, 340, 350 that may be employed according to various embodiments. For example, a sensor 330 may include a multiple axis accelerometer employed on or near muscle, particularly muscle innervated by neurons within the region of tissue being operated on. The accelerometer may be a low-g triaxial accelerometer. The accelerometer 330 may detect differential capacitance where acceleration may cause displacement of the silicon structure of the accelerometer and change its capacitance. The sensor 340 may include a strain gauge that also may be applied on or near muscle innervated by neurons within the region begin operated on. The strain gauge may a multiple planar strain gauge where the gauge's resistance or capacitance varies as a function of gauge flex forces in multiple directions. The sensor 350 may include an EMG probe. The EMG probe may include a needle to be inserted near or within muscle innervated by a neuron or neurons within the region being operated on. For example, a sensor may determine a positive response when detecting an EMG signal of about 10 to 20 μV on the EMG probe 350 for about 1 second.

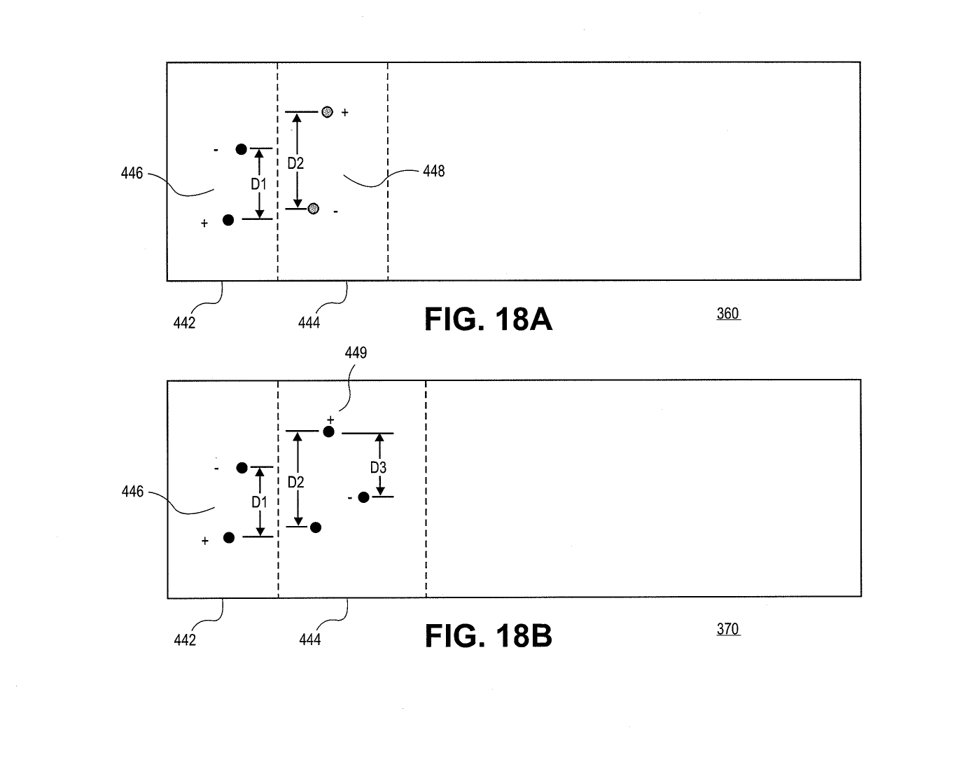

FIGS. 18A-18B illustrate the outer surface of a device having an elongate body having two regions 446, 448, wherein each region includes at least one bipole pair. The bipole pairs in the different regions may have different geometries. For example the bipole pair in the second region 444 is spaced further apart (D2>D1) than the bipole pair in the first region 442. This may result in the bipole pair in the second region projecting the bipole field further into the tissue than the bipole pair in the first region.

The configuration shown in FIG. 18B is similar, but illustrates a bipole network 449 in the second region 444 that is a tripolar electrode, having two anodes (−) separated from the cathode (+) in this example by different distances D2, D3. A bipole network may include additional cathodes and electrodes that are typically electrically coupled (e.g., to the same anodal or cathodal conductor) so that they can be stimulated substantially simultaneously.

### Methods of Operation

In general, a method of determining if a nerve is nearby a device, or a region of a device, includes the steps of exciting a bipole pair or a bipole network to pass current between the bipole pair, resulting in a limited broadcast field that can stimulate a nearby neuron. The broadcast field may be limited by the geometry of the tight bipole pairs and the bipole networks described herein, and by the applied energy. It can then be determined if a nerve has been stimulated in response to the excitation of bipole pair or network; the magnitude of the response can also be compared for different bipole networks (or bipole pairs) in different regions of the device to determine which region is nearest the nerve.

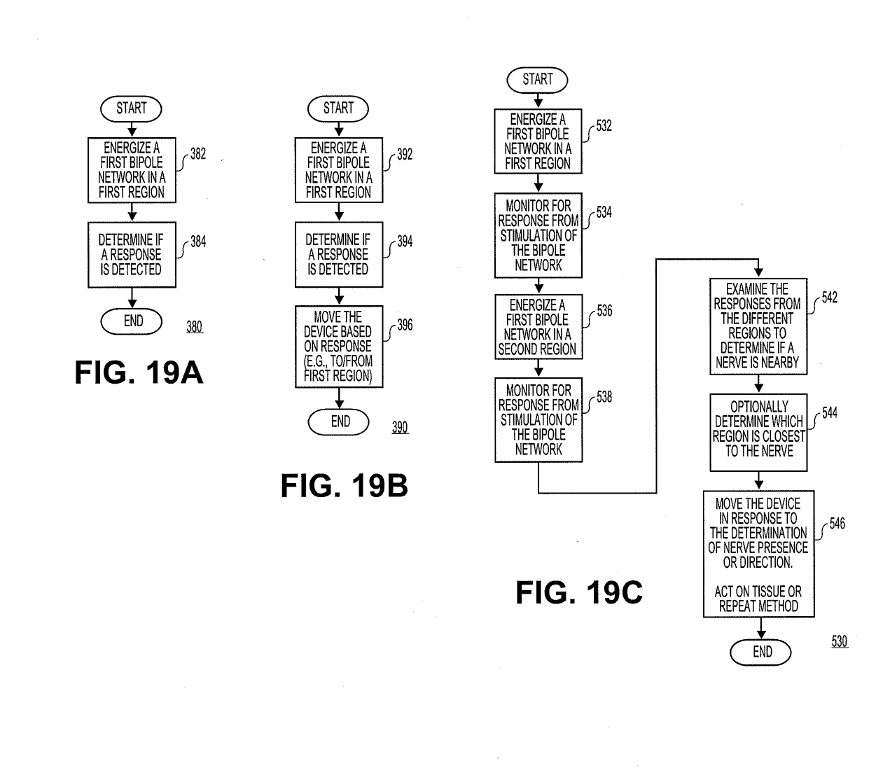

FIGS. 19A-19C are flow diagrams illustrating methods of determining if a nerve is near a device as described herein. In the algorithm 380 shown in FIG. 19A a first bipole network (or bipole pair) located on a first region or section of a device having two or more regions is energized 382. The bipole network may be energized by the application of signal for a predetermined time interval. The energization of the bipolar module may generate a current between an anode (−) and cathode (+) (or anodes and cathodes). The subject is then monitored to determine if a response is detected 384. If a response is detected, then a nerve may be nearby. The first bipole network may be energized with a first signal for a first predetermined time interval. In some variations, the first bipole network is energized as the device is moved within the tissue (e.g., as it is advanced) to continuously sense if a nerve is nearby. For example, FIG. 19B illustrates one method of sensing as advancing.

In FIG. 19B the bipole pair in the first region is energized and a response (or lack of a response) is determined. The bipole network (or pair) may be energized as described above. For example, a continuous signal may be applied, a periodic signal may be applied, or a varying (e.g., ramping) signal may be applied 392. A response may be detected by muscle twitch, nerve firing, or otherwise 394. The device can then be moved based on the response 396, or continued to be moved based on the response. Movement may be continued in the same direction (e.g., if no response is detected) or in a new direction (if a nerve is detected). Movement may also be stopped if a nerve is detected. Steps 394 and 396 may b repeated during motion to guide the device.

In some variations, multiple regions of the device are stimulated to determine if a nerve is nearby. For example, FIG. 19C illustrates one variation in which a second region of the device, having its own, separated bipole network, is stimulated. In FIG. 19C, the first bipole network (or a bipole pair) in the first region is energized 532, and the patient is monitored for a response 534 to the stimulation. The bipole pair in a second region is then energized 536, and the patient is monitored for a response 538. Additional energizing and monitoring steps (not shown) may also be included for other regions of the device, if present. The responses to the different region can be compared 542, and the device can be moved in response to the presence of a nerve in one or more of the regions 546. Optionally, it may be determined which region of the device is closer to the nerve 544. If the nerve is detected, the tissue may be acted on (e.g., cut, ablated, removed, etc., or the device may be further oriented by moving it, and these steps may be repeated. If no nerve is detected, the steps may be repeated until the device is positioned as desired, and a procedure may then be performed.

In some variations, the device may be used to position (or form a passage for) another device or a region of the device that acts on the tissue. For example, the device may be used to position a guide channel or guide wire. In some variations, the method may include repeatedly energizing only a subset of the bipole networks (or bipole pairs) until a nerve is detected, and then other bipole networks on the device may be energized to determine with more accuracy the relationship (e.g., orientation) of the nerve with respect to the device.

As mentioned, the step of monitoring or detecting a response may be performed manually (e.g., visually), or using a sensor or sensor. For example, using an accelerometer may be coupled to muscle. The accelerometer may be a multiple axis accelerometer that detects the movement of the muscle in any direction, and movement coordinated with stimulation may be detected. In some variations, a strain gauge may be used on muscle innervated by a nerve passing through or originating in the region of tissue being examined. The strain gauge may be a multiple axis strain gauge that detects the movement of the muscle in any direction. In some variations, an EMG probe may be used to measure evoked potentials of the muscle. The magnitude of any response may also be determined.

### Systems

Any of the devices described herein may be used as part of a system, which may be referred to as a nerve localization system. Systems may include components (e.g., hardware, software, or the like) to execute the methods described herein.

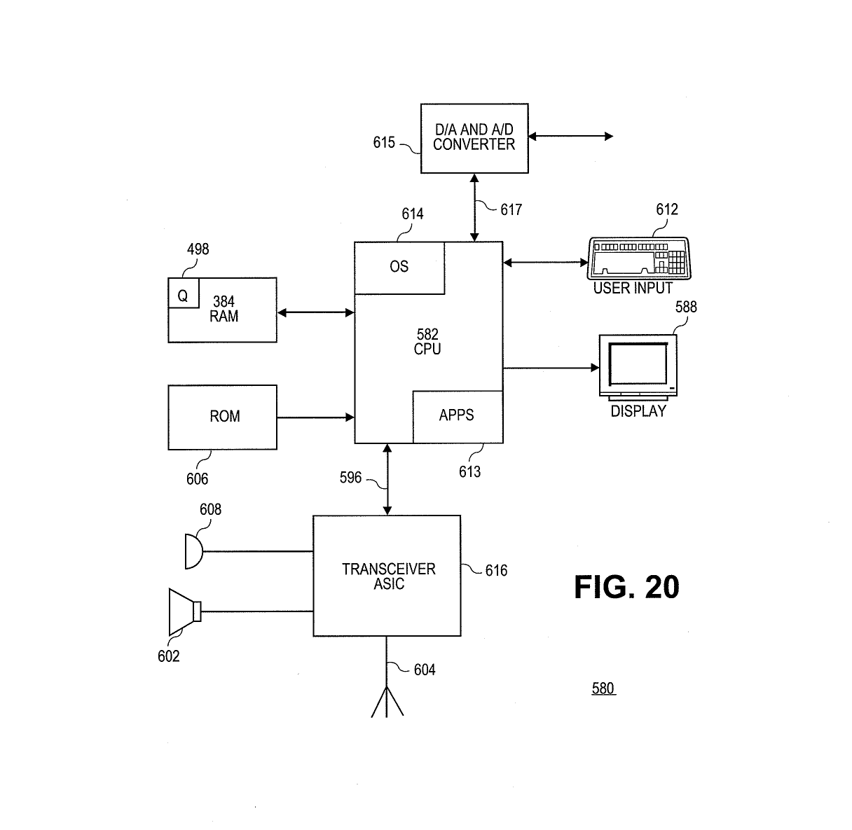

FIG. 20 is a block diagram of additional components of a system 580 for determining if a nerve is nearby a device. The components 580 shown in FIG. 20 may be used with any of the devices described herein, and may include any computing device, including a personal data assistant, cellular telephone, laptop computer, or desktop computer. The system may include a central processing unit (CPU) 582, a random access memory (RAM) 584, a read only memory (ROM”) 606, a display 588, a user input device 612, a transceiver application specific integrated circuit (ASIC) 616, a digital to analog (D/A) and analog to digital (A/D) convertor 615, a microphone 608, a speaker 602, and an antenna 604. The CPU 582 may include an OS module 614 and an application module 613. The RAM 584 may include a queue 598 where the queue 598 may store signal levels to be applied to one or more bipolar modules 46, 48. The OS module 614 and the application module 613 may be separate elements. The OS module 614 may execute a computer system or controller OS. The application module 612 may execute the applications related to the control of the system.

The ROM 606 may be coupled to the CPU 582 and may store program instructions to be executed by the CPU 582, OS module 614, and application module 613. The RAM 584 is coupled to the CPU 582 and may store temporary program data, overhead information, and the queues 598. The user input device 512 may comprise an input device such as a keypad, touch pad screen, track ball or other similar input device that allows the user to navigate through menus in order to operate the article 580. The display 588 may be an output device such as a CRT, LCD, LED or other lighting apparatus that enables the user to read, view, or hear user detectable signals.

The microphone 608 and speaker 602 may be incorporated into the device. The microphone 608 and speaker 602 may also be separated from the device. Received data may be transmitted to the CPU 582 via a serial bus 596 where the data may include signals for a bipole network. The transceiver ASIC 616 may include an instruction set necessary to communicate data, screens, or signals. The ASIC 616 may be coupled to the antenna 604 to communicate wireless messages, pages, and signal information within the signal. When a message is received by the transceiver ASIC 616, its corresponding data may be transferred to the CPU 582 via the serial bus 596. The data can include wireless protocol, overhead information, and data to be processed by the device in accordance with the methods described herein.

The D/A and A/D convertor 615 may be coupled to one or more bipole networks to generate a signal to be used to energize them. The D/A and A/D convertor 615 may also be coupled to one or more sensors 322, 324 to monitor the sensor 322, 324 state or condition.

Any of the components previously described can be implemented in a number of ways, including embodiments in software. These may include hardware circuitry, single or multi-processor circuits, memory circuits, software program modules and objects, firmware, and combinations thereof, as desired by the architect of the system 10 and as appropriate for particular implementations of various embodiments.

### Example 1

### Neural Localization when Treating Spinal Stenosis

One area of surgery which could benefit from the development of less invasive techniques including neural localization is the treatment of spinal stenosis. Spinal stenosis often occurs when nerve tissue and/or blood vessels supplying nerve tissue in the lower (or “lumbar”) spine become impinged by one or more structures pressing against them, causing pain, numbness and/or loss of function in the lower back and/or lower limb(s). In many cases, tissues such as ligamentum flavum, hypertrophied facet joint and bulging intervertebral disc impinge a nerve root as it passes from the cauda equine (the bundle of nerves that extends from the base of the spinal cord) through an intervertebral foramen (one of the side-facing channels between adjacent vertebrae). Here we provide one example of a device for determining if a nerve is nearby that may be used as part of method for treating spinal stenosis.

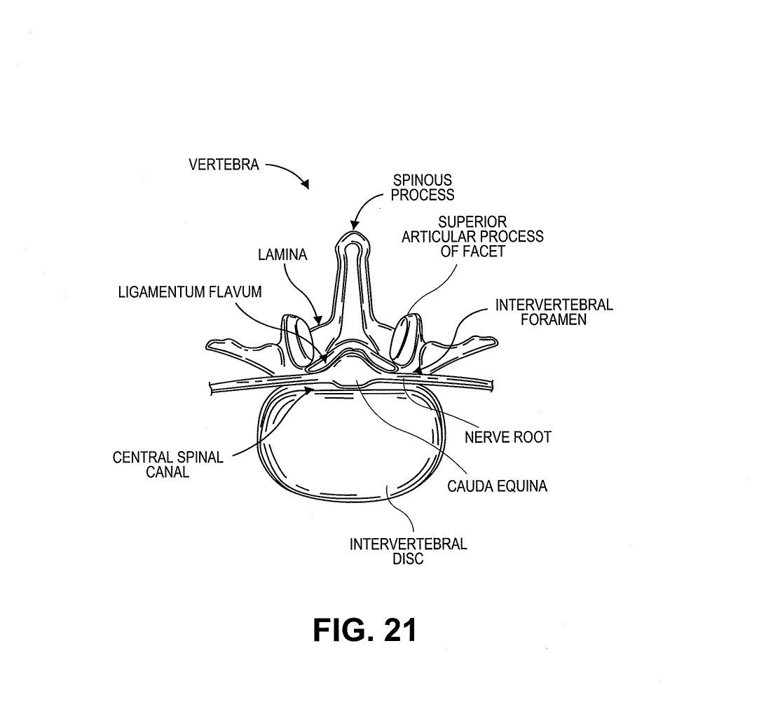

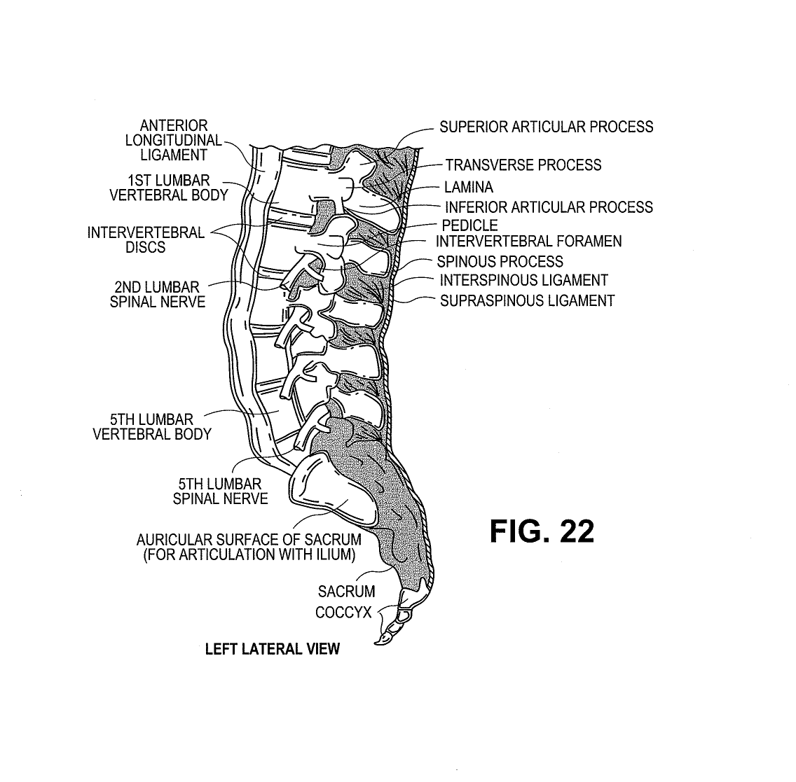

FIG. 21 is a top view of a vertebra with the cauda equina shown in cross section and two nerve roots branching from the cauda equina to exit the central spinal canal and extend through intervertebral foramina on either side of the vertebra. FIG. 22 is a side view of the lumbar spine, showing multiple vertebrae, the intervertebral foramina between adjacent vertebrae, and the 1st-5th spinal nerves exiting the foramina.

Surgery may be required to remove impinging tissue and decompress the impinged nerve tissue of a spinal stenosis. Lumbar spinal stenosis surgery typically involves first making an incision in the back and stripping muscles and supporting structures away from the spine to expose the posterior aspect of the vertebral column. Thickened ligamentum flavum is then exposed by complete or partial removal of the bony arch (lamina) covering the back of the spinal canal (laminectomy or laminotomy). In addition, the surgery often includes partial or complete facetectomy (removal of all or part of one or more facet joints), to remove impinging ligamentum flavum or bone tissue. Spinal stenosis surgery is performed under general anesthesia, and patients are usually admitted to the hospital for five to seven days after surgery, with full recovery from surgery requiring between six weeks and three months. Many patients need extended therapy at a rehabilitation facility to regain enough mobility to live independently.

Removal of vertebral bone, as in laminectomy and facetectomy, often leaves the affected area of the spine very unstable, requiring an additional highly invasive fusion procedure that puts extra demands on the patient's vertebrae and limits the patient's ability to move. Unfortunately, a surgical spine fusion results in a loss of ability to move the fused section of the back, diminishing the patient's range of motion and causing stress on the discs and facet joints of adjacent vertebral segments. Such stress on adjacent vertebrae often leads to further dysfunction of the spine, back pain, lower leg weakness or pain, and/or other symptoms. Furthermore, using current surgical techniques, gaining sufficient access to the spine to perform a laminectomy, facetectomy and spinal fusion requires dissecting through a wide incision on the back and typically causes extensive muscle damage, leading to significant post-operative pain and lengthy rehabilitation. Thus, while laminectomy, facetectomy, and spinal fusion frequently improve symptoms of neural and neurovascular impingement in the short term, these procedures are highly invasive, diminish spinal function, drastically disrupt normal anatomy, and increase long-term morbidity above levels seen in untreated patients.

A number of devices, systems and methods for less invasive treatment of spinal stenosis have been described, for example, in U.S. patent application Ser. Nos. 11/250,332, titled “DEVICES AND METHODS FOR SELECTIVE SURGICAL REMOVAL OF TISSUE,” filed on Oct. 15, 2005 now U.S. Pat. No. 7,738,968 B2; 11/375,265, titled “METHODS AND APPARATUS FOR TISSUE MODIFICATION,” filed on Mar. 13, 2006, now U.S. Pat. No. 7,887,538 B2; and 11/535,000, titled “MULTI-WIRE TISSUE CUTTER,” filed on Sep. 25, 2006, all of which applications are hereby incorporated fully be reference herein.

Challenges in developing and using less invasive or minimally invasive devices and techniques for treating neural and neurovascular impingement include accessing hard-to-reach target tissue and locating nerve tissue adjacent the target tissue, so that target tissue can be treated and damage to nerve tissue can be prevented. These challenges may prove daunting, because the tissue impinging on neural or neurovascular tissue in the spine is typically located in small, confined areas, such as intervertebral foramina, the central spinal canal and the lateral recesses of the central spinal canal, which typically have very little open space and are difficult to see without removing significant amounts of spinal bone. The assignee of the present invention has described a number of devices, systems and methods for accessing target tissue and identifying neural tissue. Exemplary embodiments are described, for example, in U.S. patent application Ser. Nos. 11/251,205, titled “DEVICES AND METHODS FOR TISSUE ACCESS,” filed on Oct. 15, 2005, now U.S. Pat. No. 7,918,849 B2; 11/457,416, titled “SPINAL ACCESS AND NEURAL LOCALIZATION,” filed on Jul. 13, 2006, now U.S. Pat. No. 7,578,819 B2; and 11/468,247, titled “TISSUE ACCESS GUIDEWIRE SYSTEM AND METHOD,” filed on Aug. 29, 2006, now U.S. Pat. No. 7,857,813 B2, all of which applications are hereby incorporated fully be reference herein.

The methods and devices for neural localization described herein may be used in less invasive spine surgery procedures, including the treatment of spinal stenosis. For example, the methods and devices described herein can be used with minimal or no direct visualization of the target or nerve tissue, such as in a percutaneous or minimally invasive small-incision procedure.

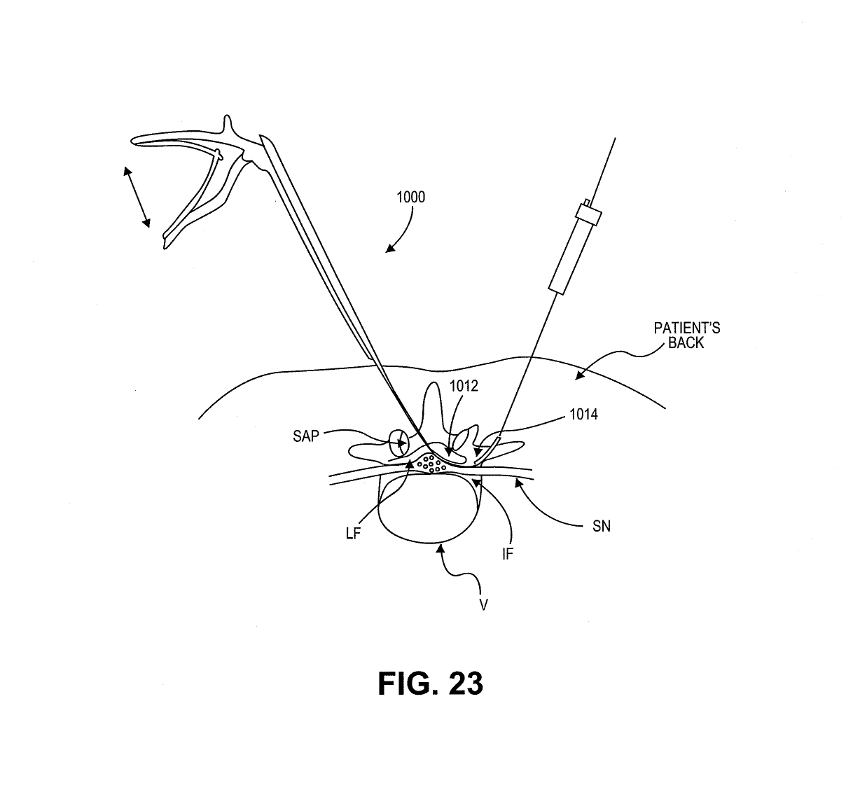

FIG. 23 illustrates one device for treatment of spinal stenosis including a tissue cutting device 1000 including a guidewire. For further explanation of guidewire systems and methods for inserting device 1000 and other tissue removal or modification devices, reference may also be made to U.S. patent application Ser. Nos. 11/468,247, titled “TISSUE ACCESS GUIDEWIRE SYSTEM AND METHOD,” filed on Aug. 29, 2006, now U.S. Pat. No. 7,857,813 B2 and 11/468,252, titled “TISSUE ACCESS GUIDEWIRE SYSTEM AND METHOD,” filed on Aug. 29, 2006, now U.S. Patent Publication No. US-2008-0086034 A1, the full disclosures of which are hereby incorporated by reference.

Cutting device 1000 may be at least partially flexible, and in some embodiments may be advanced through an intervertebral foramen IF of a patient's spine to remove ligamentum flavum LF and/or bone of a vertebra V, such as hypertrophied facet (superior articular process SAP in FIG. 23), to reduce impingement of such tissues on a spinal nerve SN and/or nerve root. In one embodiment, device 1000 cuts tissue by advancing a proximal blade 1012 on an upper side of device 1000 toward a distal blade 1014. This cutting device may be used with (or as part of) a system for determining if a nerve is nearby, and may prevent damage to nerves in the region which the device operates.

In various embodiments, device 1000 may be used in an open surgical procedure, a minimally invasive surgical procedure or a percutaneous procedure. In any procedure, it is essential for a surgeon to know that device 1000 is placed in a position to cut target tissue, such as ligament and bone, and to avoid cutting nerve tissue. In minimally invasive and percutaneous procedures, it may be difficult or impossible to directly visualize the treatment area, thus necessitating some other means for determining where target tissue and neural tissue are located relative to the tissue removal device. At least, a surgeon performing a minimally invasive or percutaneous procedure will want to confirm that the tissue cutting portion of device 1000 is not directly facing and contacting nerve tissue. The various nerve localization devices and systems described herein may help the surgeon verify such nerve/device location. A neural localization system and method may be used in conjunction with device 1000 or with any other tissue removal, tissue modification or other surgical devices. Furthermore, various embodiments may have applicability outside the spine, such as for locating nerve tissue in or near other structures, such as the prostate gland, the genitounrinary tract, the gastrointestinal tract, the heart, and various joint spaces in the body such as the knee or shoulder, or the like. Therefore, although the following description focuses on the use of embodiments of the invention in the spine, all other suitable uses for the various embodiments described herein are also contemplated.

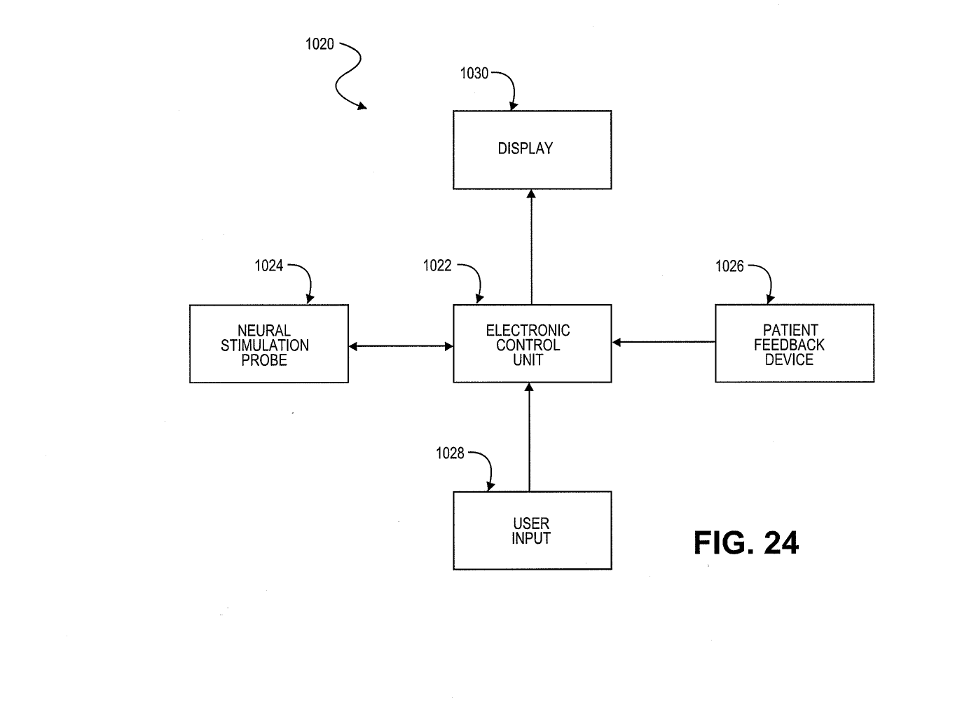

Referring now to FIG. 24, a diagrammatic representation of one embodiment of a nerve tissue localization system 1020 is shown. Neural localization system 1000 may include an electronic control unit 1024 and a neural stimulation probe 1024, a patient feedback device 1026, a user input device 1028 and a display 1030, all coupled with control unit 1022.

In one embodiment, electronic control unit (ECU) 1020 may include a computer, microprocessor or any other processor for controlling inputs and outputs to and from the other components of system 1020. In one embodiment, for example, ECU 1020 may include a central processing unit (CPU) and a Digital to Analog (D/A) and Analog to Digital Converter (A/D). ECU 1022 may include any microprocessor having sufficient processing power to control the operation of the D/A A/D converter and the other components of system 1020. Generally, ECU 1022 may control the operation of the D/A A/D converter and display device 1030, in some embodiments based on data received from a user via user input device 1028, and in other embodiments without input from the user. User input device 1028 may include any input device or combination of devices, such as but not limited to a keyboard, mouse and/or touch sensitive screen. Display device 1030 may include any output device or combination of devices controllable by ECU 1022, such as but not limited to a computer monitor, printer and/or other computer controlled display device. In one embodiment, system 1020 generates electrical signals (or other nerve stimulating energy signals in alternative embodiments), which are transmitted to electrodes on probe 1024, and receives signals from patient feedback device 1026 (or multiple feedback devices 1026 in some embodiments). Generally, ECU 1022 may generate a digital representation of signals to be transmitted by electrodes, and the D/A A/D converter may convert the digital signals to analog signals before they are transmitted to probe 1024. ECU 1022 also receive a return current from probe 1024, convert the current to a digital signal using the D/A A/D converter, and process the converted current to determine whether current was successfully delivered to the stimulating portion of probe 1024. The D/A A/D converter may convert an analog signal received by patient feedback device(s) 1026 into a digital signal that may be processed by ECU 1022. ECU 1022 may hold any suitable software for processing signals from patient feedback devices 1026, to and from probe 1024 and the like. According to various embodiments, display device 1030 may display any of a number of different outputs to a user, such as but not limited to information describing the signals transmitted to probe 1024, verification that stimulating energy was successfully delivered to a stimulating portion of probe 1024, information describing signals sensed by patient feedback devices 1026, a visual and/or auditory warning when a nerve has been stimulated, and/or the like. In various alternative embodiments, system 1020 may include additional components or a different combination or configuration of components, without departing from the scope of the present invention.

The neural stimulation probe 1024 is an elongate body having an outer surface including one or more regions with a bipole pair or bipole network. Furthermore, any suitable number of regions may be included on a given probe 1024. In various embodiments, for example, probe 1024 may includes two or more regions, each having a bipole pair or bipole network (comprising a plurality of bipole pairs) disposed along the probe in any desired configuration. In one embodiment, probe 1024 may include four regions, each having at least one bipole pairs, one pair on each of top, bottom, left and right sides of a distal portion of the probe that is configured to address neural tissue.

In some embodiments, ECU 1022 may measure current returned through probe 1024 and may process such returned current to verify that current was, in fact, successfully transmitted to a nerve stimulation portion of probe 1024. In one embodiment, if ECU 1022 cannot verify that current is being transmitted to the nerve stimulation portion of probe 1024, ECU 1022 may automatically shut off system 1020. In an alternative embodiment, if ECU 1022 cannot verify that current is being transmitted to the nerve stimulation portion of probe 1024, ECU 1022 may signal the user, via display device 1030, that probe 1024 is not functioning properly. Optionally, in some embodiments, system 1020 may include both a user signal and automatic shut-down.

Patient feedback device 1026 may include any suitable sensing device and typically includes multiple devices for positioning at multiple different locations on a patient's body. In some embodiments, for example, multiple motion sensors may be included in system 1020. Such motion sensors may include, but are not limited to, accelerometers, emitter/detector pairs, lasers, strain gauges, ultrasound transducers, capacitors, inductors, resistors, gyroscopes, and/or piezoelectric crystals. In one embodiment, where nerve tissue stimulation system 1020 is used for nerve tissue detection in the lumbar spine, feedback device 1026 may include multiple accelerometers each accelerometer attached to a separate patient coupling member, such as an adhesive pad, for coupling the accelerometers to a patient. In one such embodiment, for example, each accelerometer may be placed over a separate muscle myotome on the patients lower limbs.

When nerve tissue is stimulated by probe 1024, one or more patient feedback devices 1026 may sense a response to the stimulation and deliver a corresponding signal to ECU 1022. ECU 1022 may process such incoming signals and provide information to a user via display device 1030. For example, in one embodiment, information may be displayed to a user indicating that one sensor has sensed motion in a particular myotome. As part of the processing of signals, ECU 1022 may filter out “noise” or sensed motion that is not related to stimulation by probe 1024. In some embodiments, an algorithm may be applied by ECU 1022 to determine which of multiple sensors are sensing the largest signals, and thus to pinpoint the nerve (or nerves) stimulated by probe 1024.

In an alternative embodiment, patient feedback device 1026 may include multiple electromyography (EMG) electrodes. EMG electrodes receive EMG or evoked muscle action potential (EMAP) signals generated by muscle electrically coupled to EMG electrodes and to a depolarized nerve (motor unit). One or more nerves may be depolarized by one or more electrical signals transmitted by probe. As with the motion sensor embodiment, ECU 1022 may be programmed to process incoming information from multiple EMG electrodes and provide this processed information to a user in a useful format via display device 1030.

User input device 1028, in various embodiments, may include any suitable knob, switch, foot pedal, toggle or the like and may be directly attached to or separate and coupleable with ECU 1022. In one embodiment, for example, input device 1028 may include an on/off switch, a dial for selecting various bipolar electrode pairs on probe 1024 to stimulate, a knob for selecting an amount of energy to transmit to probe 1024 and/or the like.

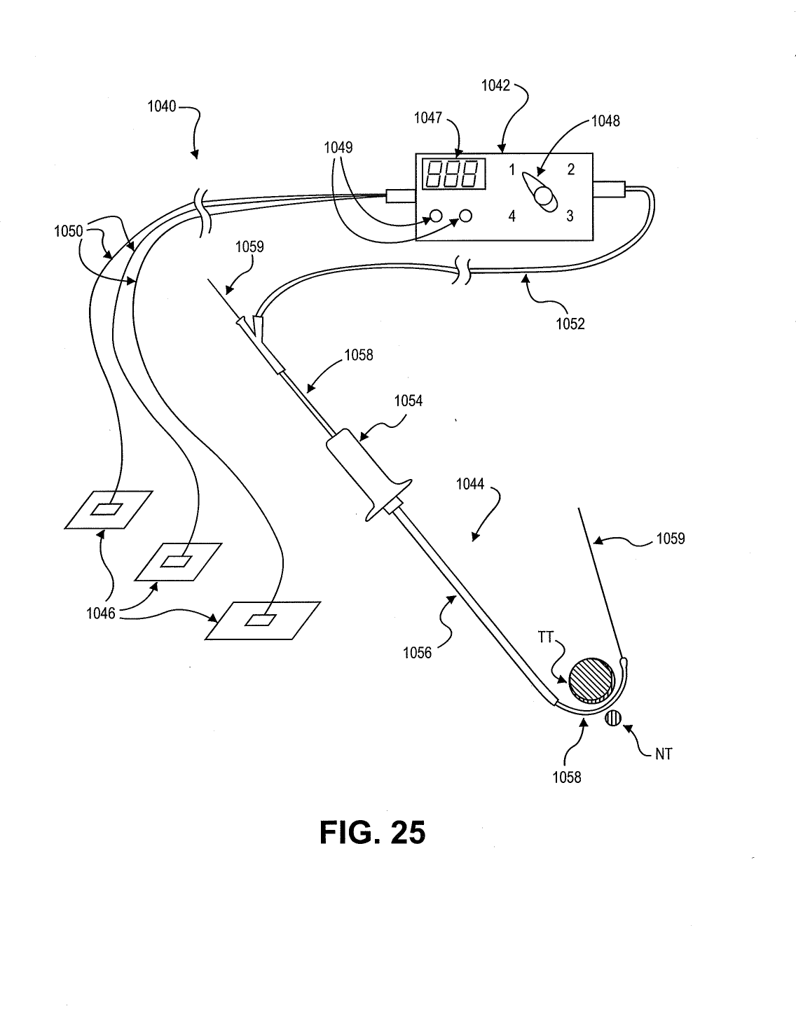

Referring now to FIG. 25, in one embodiment, a nerve tissue localization system 1040 may include an ECU 1042, a neural stimulation probe 1044, multiple patient feedback devices 1026, and a user input device 48. Probe 1044 may include, in one embodiment, a curved, flexible nerve stimulating elongate member 1058, which may slide through a rigid cannula 1056 having a handle 1054.

The probe 1044 is a device for determining if a nerve is nearby a region of the device, and includes a plurality of regions which each include one or more bipole pairs. In some variations the probe 1044 includes two regions (an upper region and a lower region), and each region includes a bipole network configured to form a continuous bipole field along the length of the probe in either the upper or lower regions. A nerve stimulating member 1058 may include a guidewire lumen for allowing passage of a guidewire 1059, for example after nerve tissue has been detected to verify that the curved portion of nerve stimulating member 1058 is in a desired location relative to target tissue TT and nerve tissue NT. Patient feedback devices 1046 and probe 1044 may be coupled with ECU 1042 via wires 1050 and 1052 or any other suitable connectors. ECU 1042 may include user input device 1048, such as a knob with four settings corresponding to top, bottom, left and right sides of a nerve tissue stimulation portion of nerve stimulating member 1058. ECU 1042 may also optionally include a display 1047, which may indicate an amount of muscle movement sensed by an accelerometer feedback device 1046. In one embodiment, ECU 1042 may include one or more additional displays, such as red and green lights 1049 indicating when it is safe or unsafe to perform a procedure or whether or not probe 1044 is functioning properly. Any other suitable displays may additionally or alternatively be provided, such as lamps, graphs, digits and/or audible signals such as buzzers or alarms.

In one embodiment, each of patient feedback devices 1046 may include an accelerometer coupled with an adhesive pad or other patient coupling device. In one embodiment, a curved portion of nerve stimulating member 1058 may be configured to pass from an epidural space of the spine at least partway through an intervertebral foramen of the spine. In other embodiments, nerve stimulating member 1058 may be straight, steerable and/or preformed to a shape other than curved.

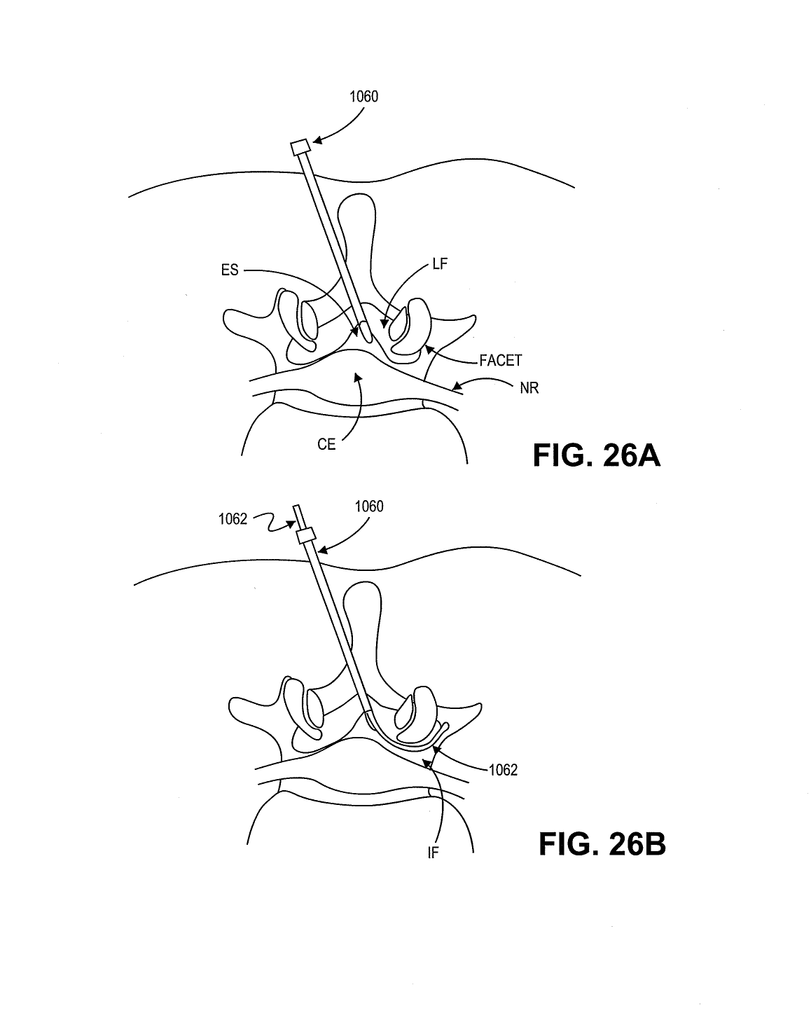

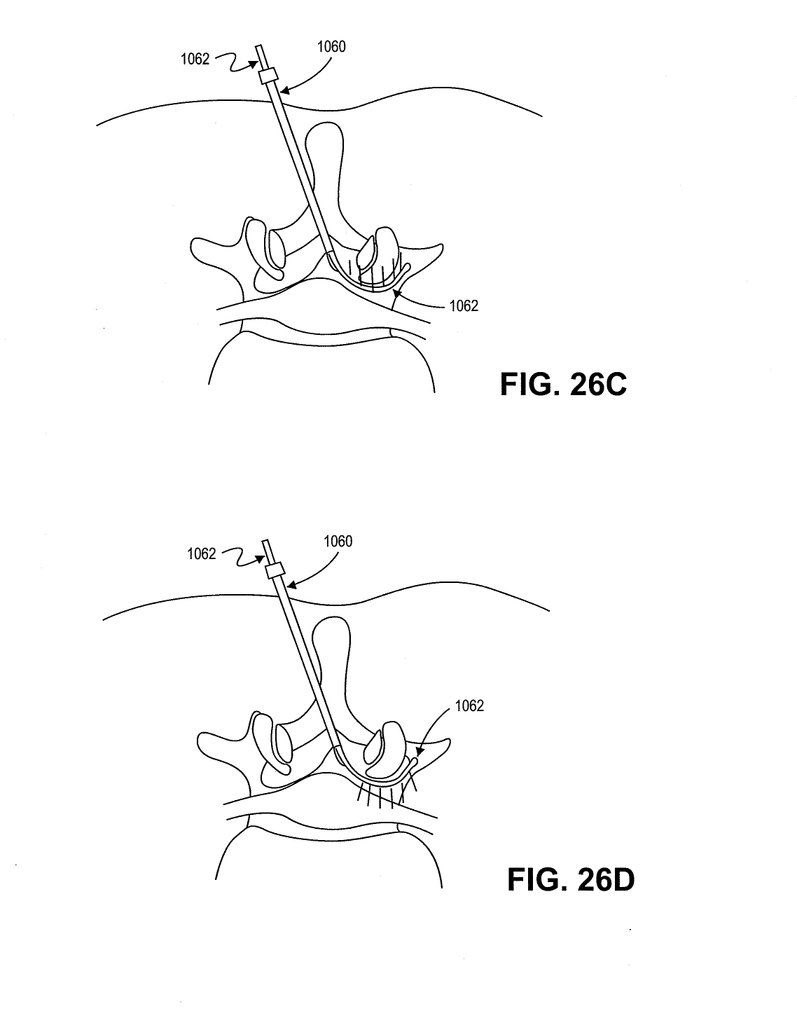

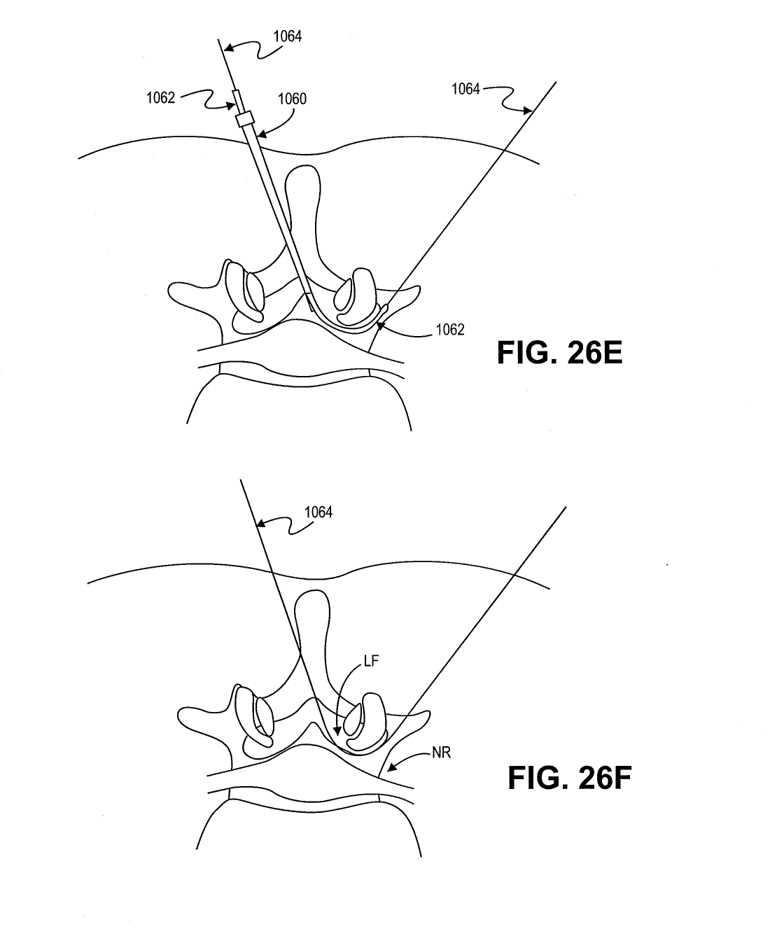

FIGS. 26A-26B and 26B describe a method for localizing nerve tissue and placing a guidewire in a desired location in a spine using the device configured to determine if a nerve is nearby. Before advancing a nerve tissue localization probe into the patient, and referring again to FIG. 25, multiple patient feedback devices 1046, such as accelerometers or EMG electrodes, may be placed on the patient, and ECU 1042 may be turned on. In one embodiment, a test current may be transmitted to probe 1044, and a return current from probe 1044 may be received and processed by ECU 1042 to verify that probe 1044 is working properly.

As shown in FIG. 26A, an epidural needle 1060 (or cannula) may be passed through the patient's skin, and a distal tip of needle 1060 may be advanced through the ligamentum flavum LF of the spine into the epidural space ES. Next, as shown in FIG. 26B, a probe that is configured to determine if a nerve is nearby the probe 1062 may be passed through epidural needle 1060, such that a curved, flexible, distal portion passes into the epidural space ES and through an intervertebral foramen IF of the spine, between target tissue (ligamentum flavum LF and/or facet bone) and non-target neural tissue (cauda equina CE and nerve root NR). As shown in FIG. 26C, the upper region of the probe having a first bipole network may be energized to generate a bipole field as current passes between the anodes and cathodes of the bipole network in the upper region 1062. In some variations, the bipole pairs may be monitored to confirm that transmitted energy returned proximally along the probe, as described previously. As shown in FIG. 26D, the lower bipole network may then be energized to generate a bipole field from the curved portion of probe 1062. In an alternative embodiment, energy may be transmitted only to the top, only to the bottom, or to the bottom first and then the top regions. In some embodiments, energy may be further transmitted to electrodes on left and right regions of probe 1062. Depending on the use of a given probe 1062 and thus its size constraints and the medical or surgical application for which it is being used, any suitable number of electrodes may form the bipole network of a particular region.

As energy is transmitted to the bipole network in any region of the probe 1062, patient response may be monitored manually or via multiple patient feedback devices (not shown in FIG. 26), such as, but not limited to, accelerometers or EMG electrodes. In one method, the same amount of energy may be transmitted to the bipole network in the different regions of the probe in series, and amounts of feedback sensed to each transmission may be measured and compared to help localize a nerve relative to probe 1062. If a first application of energy does not generate any response in the patient, a second application of energy at higher level(s) may be tried and so forth, until a general location of nerve tissue can be determined. In an alternative embodiment, the method may involve determining a threshold amount of energy required by bipole network to stimulate a response in the patient. These threshold amounts of energy may then be compared to determine a general location of the nerve relative to the probe. In another alternative embodiment, some combination of threshold and set-level testing may be used.

In one embodiment, as shown in FIG. 26E, nerve probe 1062 may include a guidewire lumen through which a guidewire may be passed, once it is determined that device 1062 is placed in a desired position between target and non-target tissue (e.g., avoiding a nerve adjacent to the upper region). As shown in FIG. 26F, when epidural needle 1060 and probe 1062 are removed, guidewire 1064 may be left in place between target tissue (such as ligamentum flavum LF and/or facet bone) and non-target tissue (such as cauda equina CE and nerve root NR). Any of a number of different minimally invasive or percutaneous surgical devices may then be pulled into the spine behind guidewire 1064 or advanced over guidewire 1064, such as the embodiment shown in FIG. 23 and others described by the assignee of the present application in other applications incorporated by reference herein.

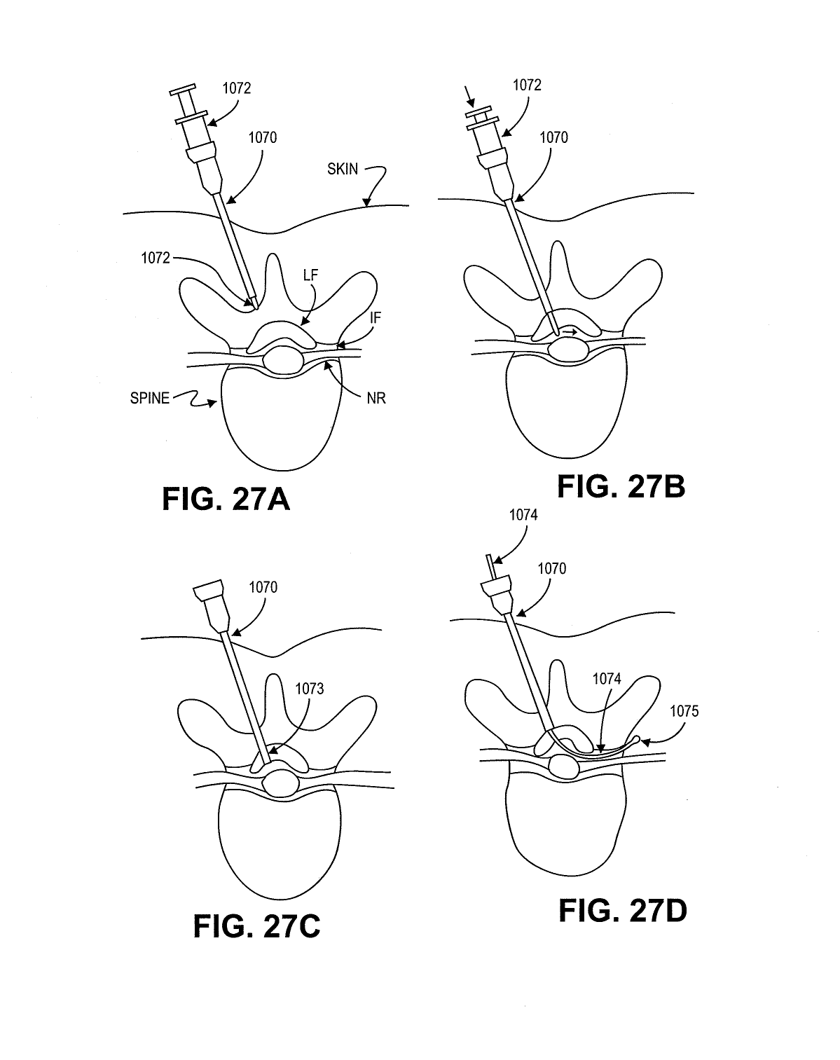

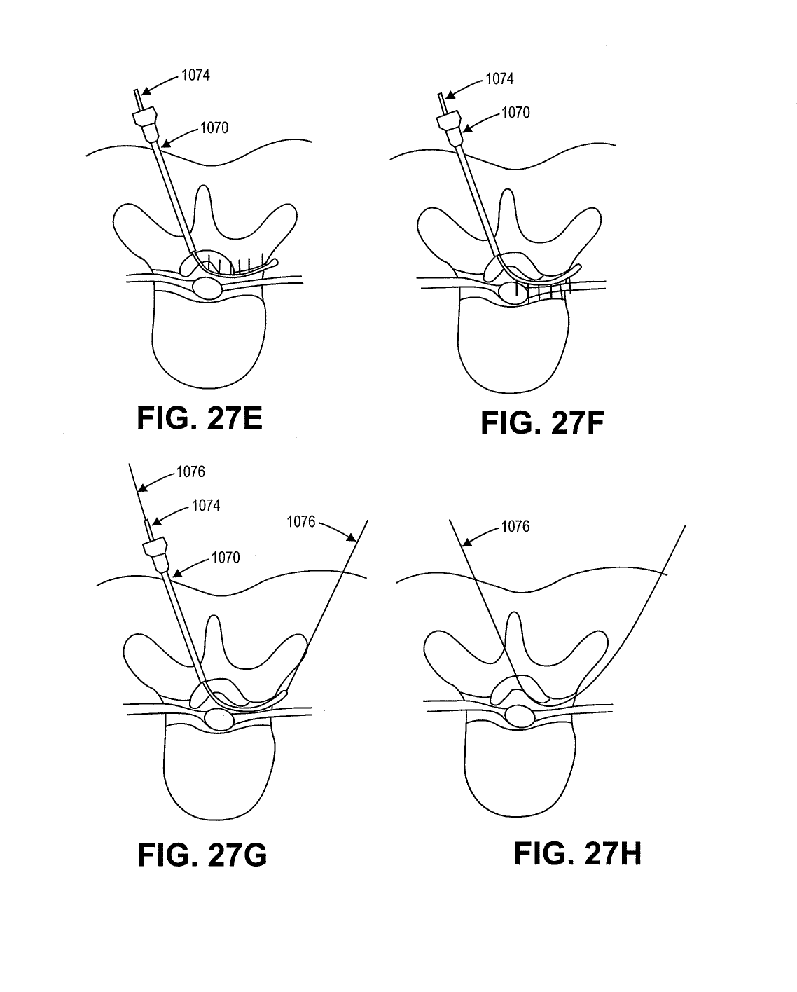

Referring now to FIGS. 27A-27H, another embodiment of a method for accessing an intervertebral foramen IF and verifying a location of a probe relative to tissue (such as ligamentum flavum LF and nerve/nerve root NR tissue) is demonstrated. In this embodiment, as shown in FIG. 27A, an access cannula 1070 may be advanced into the patient over an epidural needle 1072 with attached syringe. As shown in FIG. 27B, cannula 1070 and needle 1072 may be advanced using a loss of resistance technique, as is commonly performed to achieve access to the epidural space via an epidural needle. Using this technique, when the tip of needle 1072 enters the epidural space, the plunger on the syringe depresses easily, thus passing saline solution through the distal end of needle 1072 (see solid-tipped arrows). As shown in FIG. 27C, once epidural access is achieved, needle can be withdrawn from the patient, leaving cannula in place with its distal end contacting or near ligamentum flavum LF. Although needle 1072 may be removed, its passage through ligamentum flavum LF may leave an opening 1073 (or path, track or the like) through the ligamentum flavum LF.

As shown in FIG. 27D, a curved, flexible guide 1074 having an atraumatic distal tip 1075 may be passed through cannula 1070 and through opening 1073 in the ligamentum flavum LF, to extend at least partway through an intervertebral foramen IF. In this variation, the guide 1074 is configured as a device for determining if a nerve is nearby a region of the device. The guide 1074 is an elongate member that includes at least a first region having a bipole pair, or more preferably a bipole network thereon.

In FIG. 27E, a first bipole network on or near an external surface of guide 1074 may then be energized, and the patient may be monitored for response. As in FIG. A7F, a second bipole network disposed along guide 1074 in a different circumferential region than the region may be energized, and the patient may again be monitored for response. This process of activation and monitoring may be repeated for any number of bipole networks or as the device is manipulated in the tissue, according to various embodiments. For example, in one embodiment, guide 1074 may include a first region having a bipole network on its top side (inner curvature), a second region having a bipole network on the bottom side (outer curvature), and a third and fourth region each having a bipole network on the left side and right side, respectively. A preselected amount of electrical energy (current, voltage, and/or the like) may be transmitted to a bipole network, and the patient may be monitored for an amount of response (EMG, muscle twitch, or the like). The same (or a different) preselected amount of energy may be transmitted to a second bipole network, the patient may be monitored for an amount of response, and then optionally the same amount of energy may be transmitted sequentially to third, fourth or more bipole networks, while monitoring for amounts of response to each stimulation. The amounts of response may then be compared, and from that comparison a determination may be made as to which region is closest to nerve tissue and/or which region is farthest from nerve tissue.

In an alternative method, energy may be transmitted to a first bipole electrode and the amount may be adjusted to determine a threshold amount of energy required to elicit a patient response (EMG, muscle twitch, or the like). Energy may then be transmitted to a second bipole network, adjusted, and a threshold amount of energy determined. Again, this may be repeated for any number of bipole networks (e.g., regions). The threshold amounts of required energy may then be compared to determine the location of the regions relative to nerve tissue.

Referring now to FIG. 27G, once it is verified that guide 1074 is in a desired position relative to nerve tissue and/or target tissue, a guidewire 1076 may be passed through guide and thus through the intervertebral foramen IF and out the patient's skin. Cannula 1070 and guide 1074 may then be withdrawn, leaving guidewire 1076 in place, passing into the patient, through the intervertebral foramen, and back out of the patient. Any of a number of devices may then be pulled behind or passed over guidewire 1076 to perform a procedure in the spine.

### Rotating a Tight Bipole Pair

Another variation of nerve localizing device including one or more tight bipole pairs is a device having at least one tight bipole pair that can be scanned (e.g., rotated) over at least a portion of the circumference of the device to detect a nearby nerve.

In general, a device having a movable tight bipole pair may include an elongate body that has an outer surface and at least one bipole pair that can be scanned (moved) with respect to the outer surface of the device so as to be energized in different regions of the outer surface of the device to determine if a nerve is nearby. For example, a device may include an elongate body having an outer surface that can be divided up into a plurality of circumferential regions and a scanning that is movable with respect to the outer surface. At least one tight bipole pair (or a bipole network) is attached to the scanning surface, allowing the bipole pair or network to be scanned to different circumferential regions.

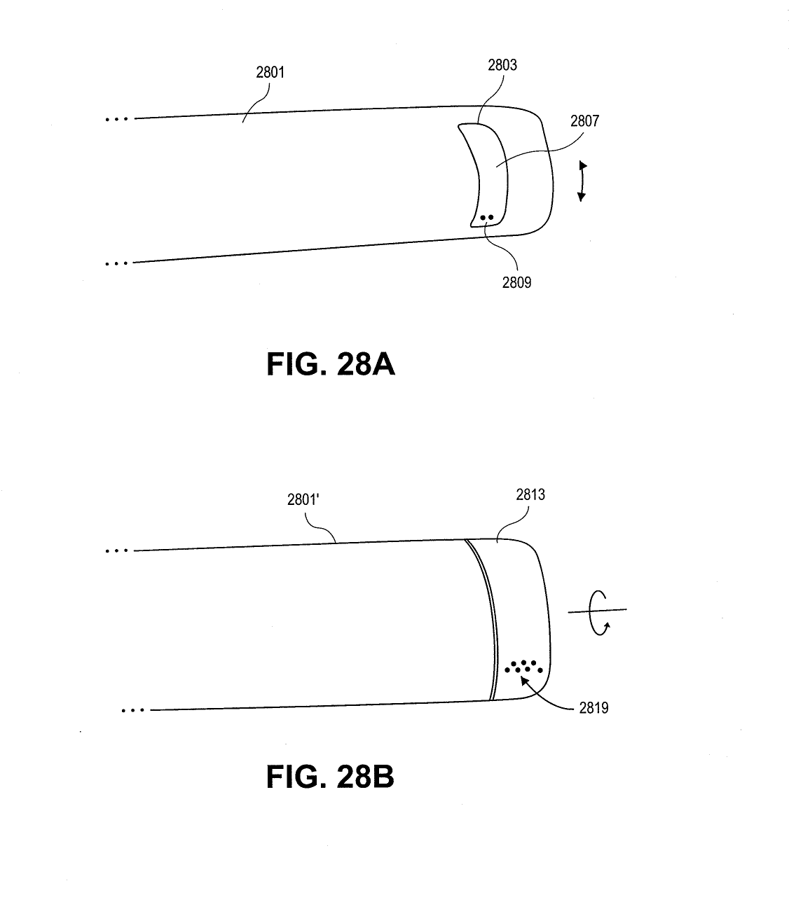

FIGS. 28A and 28B illustrate variations of a device having a scanning or movable bipole pair (or bipole network). For example, FIG. 28A includes an elongate body 2801 having an outer surface. In this variation the elongate body has a circular or oval cross-section, although other cross-sectional shapes may be used, including substantially flat. The surface of the outer body includes a window 2803 region exposing a scanning surface 2807 to which at least one bipole pair is connected. The scanning surface may be moved relative to the outer surface (as indicated by the arrow). In this example, the window extends circumferentially, and the scanning surface may be scanned radially (e.g., up and down with respect to the window).

FIG. 28B illustrates another variation, in which the distal end of the elongate body 2801′ is rotatable with respect to the more proximal region of the device. The distal end includes one or more bipole pairs. In FIG. 28 the rotatable distal end includes a bipole network 2819. The bipole network may be energized as it is rotated, or it may be rotated into different positions around the circumference of the device and energized after it has reached each position.

The devices illustrated in FIGS. 28A and 28B may include a controller configured to control the scanning (i.e., rotation) of the bipole pair. The device may also include a driver for driving the motion of the bipole pair. For example, the drive may be a motor, magnet, axel, shaft, cam, gear, etc. The controller may control the driver, and may control the circumferential position of the bipole pair (or bipole network). The device may also include an output for indicting the circumferential region of the bipole network or pair.

In operation, the scanning bipole pair can be used to determine if a nerve is near the device by moving the bipole pair or network with respect to the rest of the device (e.g., the outer surfaced of the elongate body). For example, the device may be used to determine if a nerve is nearby the device by scanning the bipole pair (or a bipolar network comprising a plurality of bipole pairs) across a plurality of circumferential regions of the outer surface of the elongate body, and by energizing the bipole pair(s) when it is in one of the circumferential regions. As mentioned, the bipole pair(s) may be energized as they are moved, or they may be energized once they are in position. The movement may be reciprocal (e.g., back and forth) or rotation, or the like.

### Tissue Manipulation Tools

Any appropriate tissue manipulation device or tool may be used with the tight bipole networks described herein, allowing the tissue manipulation devices to detect the presence of a nerve in a tissue that is to be manipulated by the device. Confirmation that a nerve either is, or is not, in a tissue that is targeted by a tissue manipulation device may be invaluable in preventing or reducing the likelihood of injury when performing procedures using the tools.

Tools that include a cavity or other tissue receiving portion are of particular interest. Such tools typically include a tissue receiving portion including at least one tissue receiving surface into which the patient's tissue will be received for manipulation. The tissue receiving surface(s) of the tool may include a tight bipole network that is configured to emit a broadcast field that is limited to the tissue receiving portion but sufficient to stimulate a nerve within the tissue receiving portion.

In practice, the tissue manipulation device may be any device that includes a tissue receiving portion which can include a tight bipole network. For example, a tissue manipulation device may include a rongeur, a scissor, a clam, a tweezers, or the like.

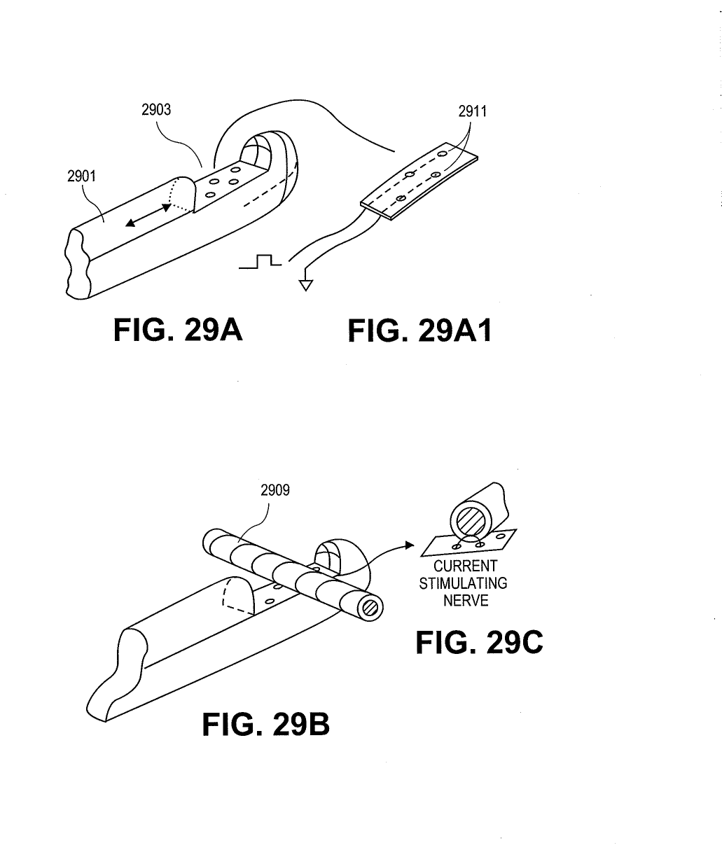

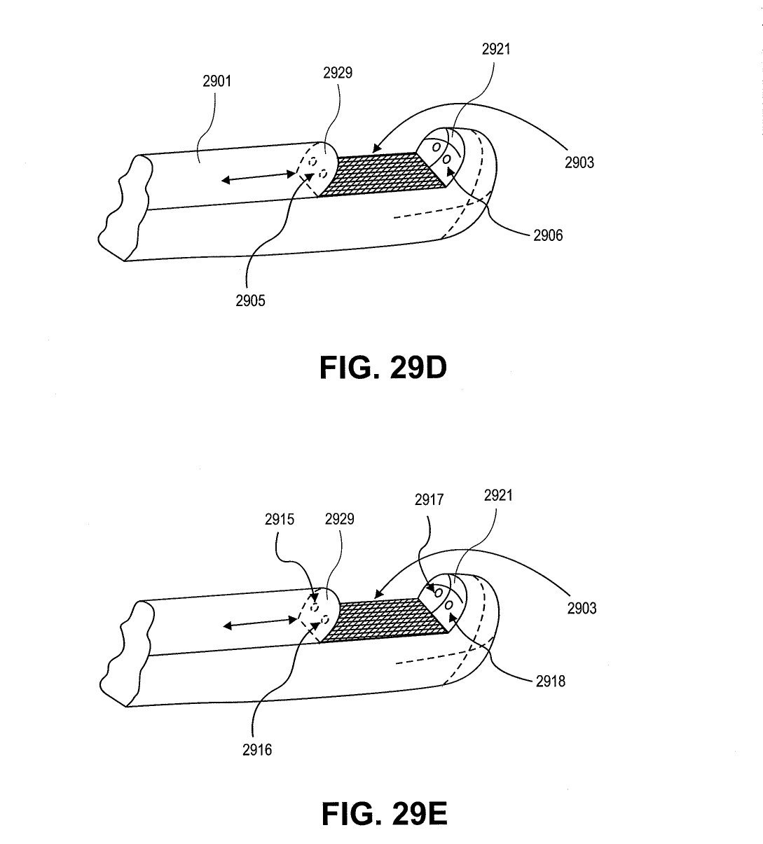

FIGS. 29A-29E (and 11D) illustrate rongeurs, one type of a tissue manipulation tool that may include a tight bipole network. In the rongeur example shown in FIGS. 29A through 29C, the device includes a tissue receiving portion 2903 configured as a mouth or cavity. The tight bipole network is arranged in the tissue receiving portion to provide feedback to a surgeon or other user that the tissue to be cut by the rongeur (in the cavity) does or does not include a nerve. In many applications the rongeur can be used for cutting through bone, ligament, and the like, as part of a procedure during which it may be undesirable to cut or damage nearby nerves.

The distal end region of the rongeur illustrated in FIGS. 29A-29E includes a blunted distal end region, and a cavity along the lateral side region of the device (oriented up in these figures), formed by a slideable biting surface 2901 that can move back and forth to bite down on tissue within the tissue receiving portion 2903. In FIG. 29A the ‘bottom’ of the tissue receiving region includes a tight bipole network arranged along the length of the bottom (e.g., in the longitudinal direction down the long axis of the device). In this example, a plurality of anodes is formed by openings to a single annodal conductor, and a plurality of cathodes is formed by opening to a single cathodal conductor. The anodes and cathodes 2911 are arranged in staggered fashion across the surface, as shown in the partial view of FIG. 29A1. In some variations the other walls forming the tissue receiving portion may also include anodes and/or cathodes forming a part of (or a complete) tight bipole network. In the example shown in FIG. 29A1, the tight bipole pairs can be formed from an insulated flex circuit.

FIGS. 29B and 29C illustrate the operation of the rongeur of FIG. 29A in use, when a nerve 2909 is present in the mouth of the device. FIG. 29C is a partial cross-section of the nerve and the tight bipole network region of the device, showing schematically a portion of the tight bipole emitted field between one of the anodes and cathodes, intersecting the nerve. Stimulation of the never by the emitted field within the tissue receiving portion of the rongeur will activate the nerve, and can be detected using one of the means described herein, including EMG, muscle twitch, or direct detection of nerve activation.

In operation, this sort of ‘smart tool’ (e.g., rongeur) can be used by first inserting it into a tissue region to be modified. For example, a rongeur that can detect the presence of a nerve in the cutting mouth can be used to cut bone or ligament within the spine as part of a spinal decompression. The tool may be inserted during an open procedure or during a minimally invasive procedure (particularly for flexible tools that may include visualization). The mouth or jaw region of the device (the tissue receiving portion) may be positioned against tissue so that the tissue is within the tissue receiving portion, and the tight bipole network may be stimulated. The patient can be simultaneously monitored for activation of a nerve from the region of the tissue in the mouth or jaw of the device. For example, if the device is used as part of a spinal decompression, an EMG or accelerometer-based system may be used to monitor for muscle twitch upon activation of the tight bipole network.

Because the tight bipole network is configured to have a controlled broadcast field that does not substantially extend beyond the mouth of the tool, activation of a nerve will only occur if the nerve is within the mouth or jaw of the device. This information may be displayed, or may be feed back to the tool to prevent it from compressing or cutting the tissue in the tissue receiving portion of the device, thereby avoiding damage to the nerve. The tight bipole network is configured to limit the emitted field, as described above. The field emitted by a tight bipole network is limited by the position and configuration of (e.g., sizes and separation between) the anode and cathode. As indicated above, the emitted field in these devices is substantially limited to the tissue receiving portion, so that only a nerve within the tissue receiving portion would be stimulated. Although some of the emitted field may escape the boundaries of the tissue receiving portion, the majority of the field is concentrated in the tissue receiving portion.

FIG. 29D shows another variation of a rongeur having a tight bipole network. The distal end region of the rongeur in FIG. 29D is structurally similar to the rongeur shown in FIGS. 29A-29C, however the tight bipole network is arranged differently. In this example, the two side surfaces of the tissue receiving portion each include a tight bipole pair 2905, 2906. One of the side surfaces 2923 is the surface of the movable biting member 2905 that faces into the tissue receiving portion. The opposite wall 2921 is stationary relative to the biting surface 2901. Thus, the opposite walls 2921, 2923 of the tissue receiving portion each have at least one bipole pair forming the bipole network.

FIG. 29E shows a similar variation, in which the anodes and cathodes of the tight bipole network are on opposite walls 2921, 2923 of the tissue receiving portion. In this example, the anodes 2915, 2916 are on the movable biting member 2905, and the cathodes 2917, 2918 are on the opposite wall 2921. In some variations both the opposite walls and the bottom of the tissue receiving portion (e.g., all of the surfaces of the tissue receiving portion) may have anodes and/or cathodes of the tight bipole network.

### Systems for Controlling Tools

As described above, and illustrated in FIGS. 17A and 17B, an accelerometer-based detection system may be used to determine when a nerve has been stimulated. An accelerometer-based system for determining if a nerve is nearby a tool having a neurostimulation electrode may be used with any appropriate neurostimulation electrode, and is not limited to the tight bipole pair devices and systems that are described herein. Thus, an accelerometer-based system may be used with a monopolar neurostimulation electrode, a bipolar neurostimulation electrode, or a multipolar neurostimulation electrode, as well as the tight bipole networks described above.

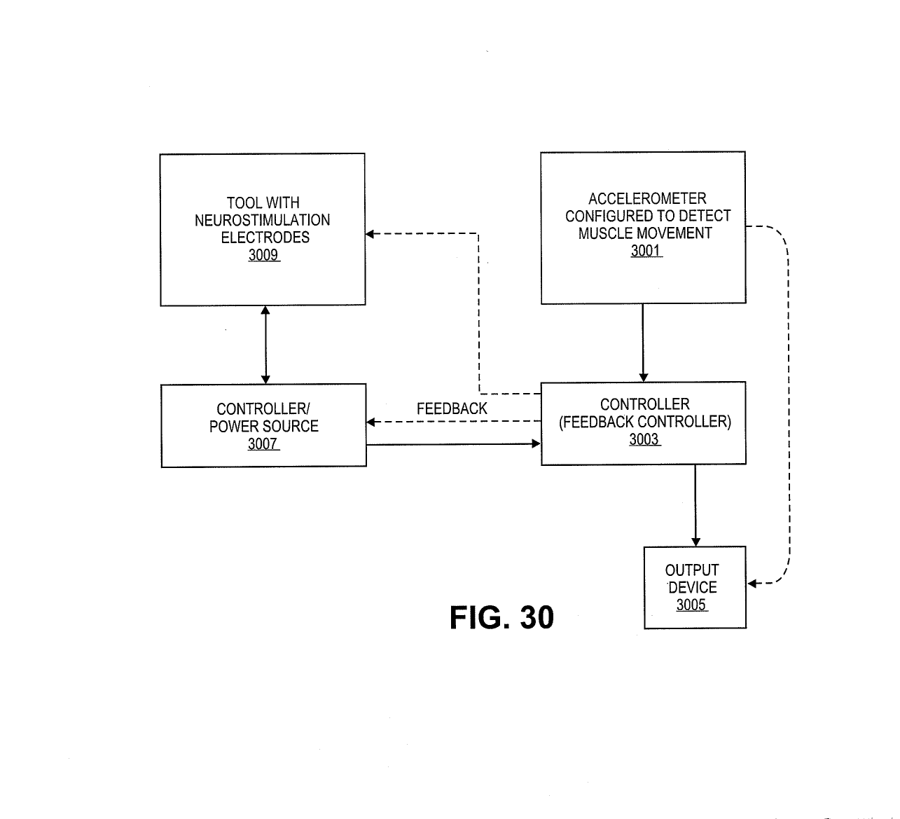

In general, an accelerometer-based detection system for determining if a nerve is nearby an insertable tool having a neurostimulation electrode includes an accelerometer that is configured to detect muscle twitch, a feedback controller, and a tool having at least one neurostimulation electrode. FIG. 30 schematically illustrates these elements, as well as other optional features.

In FIG. 30, the accelerometer configured to detect muscle twitch 3001 is shown connected to a feedback controller 3003. Any appropriate accelerometer may be used, including low-g triaxial accelerometers, as mentioned above. More than one accelerometer may be used. These accelerometers may be adapted or configured specifically for detection of muscle twitch by including filtering or sensitivity adjustment. For example, the accelerometers may be filtered to prevent low-frequency stimulation that may result from movement artifact not linked to stimulation by the neurostimulation electrode. The signal output from the accelerometer(s) may be processed on-board the accelerometer 3001, or may be processed within the feedback controller 3003. In some variations, the feedback controller is integrated with the accelerometer(s).

The accelerometers are typically secured to the patient, and may be secured to the outside of the patient (e.g., the skin of the patient, or a garment worn by the patient, etc.). In some variations, the accelerometer is implanted within the patient.

The feedback controller 3003 receives output from the accelerometer, and may also receive output from the controller/power source 3007 for the neurostimulation electrode on the insertable tool. The controller 3003 may coordinate this input to determine if stimulation by the neurostimulation electrode has resulted in muscle twitch. For example, the controller may compare the timing of the applied neurostimulation and any detected muscle twitch. In some variations the neurostimulation may be applied in a pattern (e.g., duration on/duration off) that may be compared to the pattern of detected muscle twitch by the controller 3003. This comparison may confirm the activation of a nerve, and therefore confirm that a nerve is being activated by the neurostimulation electrode. The result of any processing by the feedback controller may be output. For example, signals from the feedback controller may be visually output. A display or monitor may indicate activation of a nerve by the neurostimulation electrode. In some variations, the output is a light (e.g., an LED or other color-coded signal) indicating stimulation of the nerve. Multiple neurostimulation electrodes may be used, and the feedback controller may indicate (via output) nerve activation relative to each neurostimulation electrode. In some variations, the output from the controller 3003 may be audible, from a speaker or speakers. For example, the output may buzz or otherwise indicate proximity to a nerve. More than one output modality may be used. In some variations the signal of the accelerometer(s) may be directly output.

Accelerometer-based systems for detecting neurostimulation described herein may be advantageous over comparable EMG systems, since they do not require the electronic amplification systems and technical expertise needed for use with comparable EMG systems. EMG systems typically require recording and analysis of EMG signals during or following neurostimulation. This analysis is typically done by a person trained to interpret the often complex EMG signals. In contrast the output of the accelerometer (sensing muscle twitch) may be readily output and understood without requiring a technician to interpret the output.

The system may also include feedback that helps control the insertable tool. In addition to the output seen, heard, or otherwise sensed by a user manipulating a tool having a neurostimulation electrode, the feedback controller may send data or control signals back to the tool to regulate its activity. For example, if the tool is a cutting or biting tool such as the rongeurs described above, a signal from the feedback controller indicating that a nerve has been detected may be sent to the tool (or a controller for the tool) to prevent it from cutting or compressing the tissue, thereby protecting the sensed nerve from damage. As another example, the tool may be a probe or hook (e.g., a love hook) to be used to manipulate the nerve (e.g., by pushing or protecting it. Feedback from the feedback controller 3003 may be used to activate the probe or hook, allowing it to move and thereby manipulate the nerve. The tool may also be a therapy-delivery device that is activated when in proximity to a target nerve. Feedback from the accelerometer-based system may trigger the release of the therapy. In one example, the therapy is a drug to be delivered.

The examples and illustrations included herein show, by way of illustration and not of limitation, specific embodiments in which the subject matter may be practiced. Other embodiments may be utilized and derived therefrom, such that structural and logical substitutions and changes may be made without departing from the scope of this disclosure. Such embodiments of the inventive subject matter may be referred to herein individually or collectively by the term “invention” merely for convenience and without intending to voluntarily limit the scope of this application to any single invention or inventive concept, if more than one is in fact disclosed. Thus, although specific embodiments have been illustrated and described herein, any arrangement calculated to achieve the same purpose may be substituted for the specific embodiments shown. This disclosure is intended to cover any and all adaptations or variations of various embodiments. Combinations of the above embodiments, and other embodiments not specifically described herein, will be apparent to those of skill in the art upon reviewing the above description.

What is claimed is:

1. A tissue manipulation device that can detect the presence of a nerve in a tissue to be manipulated by the device, the device comprising:
a tissue receiving portion including a first tissue receiving surface and a second tissue receiving surface, wherein the first tissue receiving surface is configured to move relative to the second tissue receiving surface to engage tissue within the tissue receiving portion; and
a tight bipole network in communication with the tissue receiving portion, wherein the tight bipole network is configured to emit a broadcast field that is limited to the tissue receiving portion and sufficient to stimulate a nerve within the tissue receiving portion.

2. The tissue manipulation device of claim 1 further comprising a handle proximal to the tissue receiving portion.

3. The tissue manipulation device of claim 2, wherein the handle comprises a control for moving the first tissue receiving surface.

4. The tissue manipulation device of claim 1 further comprising an elongate body extending proximally to the tissue receiving portion.

5. The tissue manipulation device of claim 1, wherein the tissue receiving portion comprises a jaw.

6. The tissue manipulation device of claim 1, wherein the second tissue receiving surface is not movable.

7. The tissue manipulation device of claim 1, wherein the tight bipole network comprises a bipole pair.

8. The tissue manipulation device of claim 1, wherein the tight bipole network comprises a plurality of anodes and cathodes arranged within the tissue receiving portion.

9. The tissue manipulation device of claim 1, wherein the tight bipole network comprises a plurality of anodes and cathodes configured to form an effectively continuous bipole field within the tissue receiving portion.

10. The tissue manipulation device of claim 1, wherein the tissue manipulation device is configured as a rongeur and the first tissue receiving surface is configured to move relative to the second tissue receiving surface to cut tissue within the tissue receiving portion.

11. A rongeur device for cutting tissue that can detect the presence of a nerve in the tissue to be cut, the rongeur device comprising:
a jaw having a tissue receiving portion, the tissue receiving portion including a first tissue receiving surface and a second tissue receiving surface, wherein the first tissue receiving surface is configured to move towards the second tissue receiving surface to cut tissue within the tissue receiving portion; and
a tight bipole network on the jaw configured to emit a broadcast field that is limited to the tissue receiving portion and sufficient to stimulate a nerve within the tissue receiving portion.

12. The rongeur device of claim 11 further comprising a handle.

13. The rongeur device of claim 11 further comprising an elongate body, wherein the jaw is located at the distal region of the elongate body.

14. The rongeur device of claim 11, wherein the second tissue receiving surface is not movable.

15. The rongeur device of claim 11, wherein the tight bipole network comprises a bipole pair.

16. The rongeur device of claim 11, wherein the tight bipole network comprises a plurality of anodes and cathodes arranged within the tissue receiving portion.

17. The rongeur device of claim 11, wherein the tight bipole network comprises a plurality of anodes and cathodes configured to form an effectively continuous bipole field within the tissue receiving portion.

18. A rongeur device for cutting tissue that can detect the presence of a nerve in the tissue to be cut, the rongeur device comprising:
a handle;
an elongate body extending distally from the handle along a longitudinal axis;
a tissue receiving portion near the distal end of the elongate body, the tissue receiving portion including a first tissue receiving surface and a second tissue receiving surface, wherein the first tissue receiving surface is configured to move longitudinally towards the second tissue receiving surface to cut tissue within the tissue receiving portion; and
a tight bipole network in communication with the tissue receiving portion wherein the tight bipole network is configured to emit a broadcast field that is limited to the tissue receiving portion and sufficient to stimulate a nerve within the tissue receiving portion.

19. The rongeur device of claim 18, wherein the second tissue receiving surface is not movable.

20. The rongeur device of claim 18, wherein the tight bipole network comprises a bipole pair.

21. The rongeur device of claim 18, wherein the tight bipole network comprises a plurality of anodes and cathodes arranged within the tissue receiving portion.

22. The rongeur device of claim 18, wherein the tight bipole network comprises a plurality of anodes and cathodes configured to form an effectively continuous bipole field within the tissue receiving portion.

23. A method of cutting tissue using a rongeur device capable of determining if a nerve is present in the tissue to be cut, the method comprising
placing tissue within a tissue receiving portion of the rongeur device;
energizing a tight bipole network to emit a broadcast field that is substantially limited to the tissue receiving portion;
determining if a nerve or a portion of a nerve is present in the tissue receiving portion of the rongeur device; and
cutting the tissue within the tissue receiving portion of the rongeur device.

24. The method of claim 23, wherein the step of energizing the tight bipole network comprises applying energy to a plurality of bipole pairs in communication with the tissue receiving portion of the rongeur device.

25. The method of claim 23, wherein the step of energizing the tight bipole network comprises emitting an effectively continuous bipole field within the tissue receiving portion of the rongeur device.

26. The method of claim 23, wherein the step of determining if a nerve or portion of a nerve is present comprises observing an EMG.

27. The method of claim 23, wherein the step of determining if a nerve or portion of a nerve is present comprises monitoring muscle twitch.

28. The method of claim 23, wherein the step of cutting comprises actuating the handle of the rongeur device to move a first tissue receiving surface of the tissue receiving portion of the rongeur device towards a second tissue receiving surface.

29. The method of claim 23, wherein the step of cutting comprises cutting the tissue within the tissue receiving portion of the rongeur device if a nerve or portion of a nerve is not present in the tissue receiving portion of the rongeur device.
